SEQUENCE LISTING (3 sequences)

SEQ ID NO: 1 - DNA, 30 bp, Artificial Sequence
  Description of Artificial Sequence Synthetic oligonucleotide
gtactcaatgaattaggtggaagaccgagg

SEQ ID NO: 2 - protein, 4 aa, Artificial Sequence
  Description of Artificial Sequence Synthetic peptide
DEAD

SEQ ID NO: 3 - protein, 4 aa, Artificial Sequence
  Description of Artificial Sequence Synthetic peptide
DEAH

(19) United States
(12) Patent Application Publication
(10) Pub. No.: US 2007/0122826 A1
(43) Pub. Date: May 31, 2007

(54) Minimal bacterial genome

(75) Inventors: John I. Glass, Germantown, MD (US); Hamilton O. Smith, Reisterstown, MD (US); Clyde A. Hutchison III, Rockville, MD (US); Nina Y. Alperovich, Germantown, MD (US); Nacyra Assad-Garcia, Rockville, MD (US)

Correspondence Address: VENABLE LLP, P.O. BOX 34385, WASHINGTON, DC, 20043-9998, US

(73) Assignee: J. Craig Venter Institute, Inc., Rockville, MD (US)

(21) Appl. No.: 11/546,364

(22) Filed: Oct. 12, 2006

Related U.S. Application Data
(60) Provisional application No. 60/725,295, filed on Oct. 12, 2005.

(51) Int. Cl. C40B 30/06; C40B 40/10; C07H 21/04; C12P 7/06; C12P 1/06; C12N 9/02; C12N 1/21

(52) U.S. Cl. 435/6; 435/161; 435/169; 435/252.3; 435/189; 536/23.2

(57) ABSTRACT

The present invention relates, e.g., to a minimal set of protein-coding genes which provides the information required for replication of a free-living organism in a rich bacterial culture medium, wherein (1) the gene set does not comprise the 101 genes listed in Table 2; and/or wherein (2) the gene set comprises the 381 protein-coding genes listed in Table 3 and, optionally, one of more of: a set of three genes encoding ABC transporters for phosphate import (genes MG410, MG411 and MG412; or genes MG289, MG290 and MG291); the lipoprotein-encoding gene MG185 or MG260; and/or the glycerophosphoryl diester phosphodiesterase gene MG293 or MG385.

This application claims the benefit of the filing date of U.S. provisional application 60/725,295, filed Oct. 12, 2005, which is incorporated by reference herein in its entirety.

Aspects of this invention were made with government support (DOE grant number DE-FG02-02ER63453). The government has certain rights in the invention.

### FIELD OF THE INVENTION

This invention relates, e.g., to the identification of non-essential genes of bacteria, and of a minimal set of genes required to support viability of a free-living organism.

### BACKGROUND INFORMATION

One consequence of progress in the new field of synthetic biology is an emerging view of cells as assemblages of parts that can be put together to produce an organism with a desired phenotype (1). That perspective begs the question: “How few parts would it take to construct a cell?” In an environment that is free from stress and provides all necessary nutrients, what would comprise the simplest free-living organism? This problem has been approached theoretically and experimentally in our laboratory and elsewhere.

In a comparison of the first two bacterial genomes sequenced, Mushegian and Koonin projected that the 256 orthologous genes shared by the Gram negative Haemophilus influenzae and the Gram positive M genitalium genomes are a close approximation of a minimal gene set for bacterial life (2). More recently Gil et al. proposed a 206 protein-coding gene core of a minimal bacterial gene set based on analysis of several free-living and endosymbiotic bacterial genomes (3).

In 1999 some of the present inventors reported the first use of global transposon mutagenesis to experimentally determine the genes not essential for laboratory growth of M genitalium (4). Since then there have been numerous other experimental determinations of bacterial essential gene sets using our approach and other methods such as site directed gene knockouts and antisense RNA (5-12). Most of these studies were done with human pathogens, often with the aim of identifying essential genes that might be used as antibiotic targets. Almost all of these organisms contain relatively large genomes that include many paralogous gene families. Disruption or deletion of such genes shows they are non-essential but does not determine if their products perform essential biological functions. It is only through gene essentiality studies of bacteria that have near minimal genomes that we bring empirical verification to the compositions of hypothetical minimal gene sets.

The Mollicutes, generically known as the mycoplasmas, are an excellent experimental platform for experimentally defining a minimal gene set. These wall-less bacteria evolved from more conventional progenitors in the Firmicutes taxon by a process of massive genome reduction. Mycoplasmas are obligate parasites that live in relatively unchanging niches requiring little adaptive capability. M genitalium , a human urogenital pathogen, is the extreme manifestation of this genomic parsimony, having only 482 protein-coding genes and the smallest genome at ˜580 kb of any known free-living organism capable of being grown in pure culture (13). The bacteria can grow independently on an agar plate free of other living cells. While more conventional bacteria with larger genomes used in gene essentiality studies have on average 26% of their genes in paralogous gene families, M genitalium has only 6% (Table 1). Thus, with its lack of genomic redundancy and contingencies for different environmental conditions, M genitalium is already close to being a minimal bacterial cell.

The 1999 report by some of the present inventors on the essential microbial gene for M genitalium and its closest relative, Mycoplasma pneumoniae, mapped ˜2200 transposon insertion sites in these two species, and identified 130 putatively non-essential M genitalium protein-coding genes or M pneumoniae orthologs of M genitalium genes. In that report (Hutchison et al. (1999) Science 286, 2165-9), those authors estimated that 265 to 350 of the protein-coding genes of M genitalium are essential under laboratory growth conditions (4). However proof of gene dispensability requires isolation and characterization of pure clonal populations, which they did not do. In that report, the authors grew Tn4001 transformed cells in mixed pools for several weeks, and then isolated genomic DNA from those mixtures of mutants. They sequenced amplicons from inverse PCRs using that DNA as a template to identify the transposon insertion sites in the mycoplasma genomes. Most of the genes containing transposon insertions encoded either hypothetical proteins or other proteins not expected to be essential. Nonetheless, some of the putatively disrupted genes, such as isoleucyl and tyrosyl-tRNA synthetases (MG345 & MG455), DNA replication gene dnaA (MG469), and DNA polymerase III, subunit alpha (MG261) are thought to perform essential functions. They hypothesized how genes generally thought to be essential might be disrupted: a gene may be tolerant of the transposon insertion and not actually disrupted, cells could contain two copies of a gene, or the gene product may be supplied by other cells in the same mixed pool of mutants.

Disclosed herein is an expanded study in which we have isolated and characterized M genitalium Tn4001 insertion mutants that were present in individual colonies picked from agar plates. This analysis has provided a new, more thorough, estimate of the number of essential genes in this minimalist bacterium.

### DESCRIPTION OF THE DRAWINGS

FIG. 1 shows the accumulation of new disrupted M genitalium genes (top line, thick) and new transposon insertion sites in the genome (bottom line, thin) as a function of the total number of analyzed primary colonies and subcolonies with insertion sites different from that of the parental primary colony.

FIGS. 2A-21 show global transposon mutagenesis of M genitalium . The locations of transposon insertions from the current study are noted by a Δ below the insertion site on the map. The letters over the Gene Loci (MG###) refer to the functional category of the gene product as listed.

| | |
|---|---|
| A | Biosynthesis of cofactors, prosthetic grps, and |
| | carriers |
| B | Purines, pyrimidines, nucleosides, and |
| | nucleotides |
| C | Cell envelope |
| D | Cellular processes |
| E | Central intermediary metabolism |
| F | DNA metabolism |
| G | Energy metabolism |
| H | Fatty acid and phospholipid metabolism |
| I | Hypothetical proteins |
| J | Protein fate |
| K | Protein synthesis |
| L | Regulatory functions |
| M | Transcription |
| N | Transport and binding proteins |
| X | Unknown function |
| P | cell/organism defense |
| R | rRNA and tRNA genes |

FIG. 3 shows the frequency of Tn4001 tet insertions. These histograms show the frequency we identified mutants with transposon insertions at different sites in the genome. The abscissa is the M genitalium genome site where the transposon inserts. Some mutations proved to be highly prone to transposon migration. In subcolonies with insertion sites different than the primary clone there was a preference to jump to a region of the genome from ˜350,000 to 500,000 base pairs rich in topological features such as pallindromic regions and cruciform elements (van Noort et al. (2003) Trends Genet 19, 365-369).

FIG. 4 shows metabolic pathways and substrate transport mechanisms encoded by M genitalium . White letters on black boxes mark non-essential functions or proteins based on our current gene disruption study. Question marks denote enzymes or transporters not identified that would be necessary to complete pathways, and those missing enzyme and transporter names are italicized. Transporters are drawn spanning the cell membrane. The arrows indicate the predicted direction of substrate transport. The ABC type transporters are drawn with a rectangle for the substrate-binding protein, diamonds for the membrane-spanning permeases, and circles for the ATP-binding subunits.

### DESCRIPTION OF THE INVENTION

The inventors have identified 101 protein-coding genes that are non-essential for sustaining the growth of an organism, such as a bacterium, in a rich bacterial culture medium, e.g. SP4. Such a culture medium contains all of the salts, growth factors, nutrients etc. required for bacterial growth under laboratory conditions. A minimal set of genes required for sustaining the viability of a free-living organism under laboratory conditions is extrapolated from the identification of these non-essential genes. By a “minimal gene set” is meant the minimal set of genes whose expression allows the viability (e.g., survival, growth, replication, proliferation, etc.) of a free-living organism in a particular rich bacterial medium as discussed above.

The 101 protein-coding genes of M genitalium that were disrupted in the bacteria and nevertheless retained viability, and are thus dispensable (non-essential) for growth, are listed in Table 2, where they are grouped by their functional roles. The 381 genes that were not disrupted are summarized in Table 3, where they are also grouped by functional roles. These genes form part of a minimal essential gene set. Other genes may also be part of a minimal gene set. At minimum, these other genes include protein-coding genes for ABC transporters for phosphate and/or phosphonate, and certain lipoproteins and/or glycerophosphoryl diester phosphodiesterases; and RNA-encoding genes.

As noted above, the some of the present inventors published a preliminary study in 1999 that reported putative sets of genes that appeared to be either essential or disposable for viability. Table 4 lists genes identified in the present study as being dispensable, but which were not so identified in the 1999 paper. Table 5 lists genes identified in the present study as being required for growth, but which were not so identified in the 1999 paper.

One aspect of the invention is a set of protein-coding genes that provides the information required for replication of a free-living organism under axenic conditions in a rich bacterial culture medium, such as SP4, (e.g., a minimal set of protein-coding genes),

wherein the gene set lacks at least 40 of the 101 protein-coding genes listed in Table 2 (the “lacking genes”), or functional equivalents thereof, wherein at least one of the genes in Table 4 is among the lacking genes;

wherein the set comprises between 350 and 381 of the 381 protein-coding genes listed in Table 3, or functional equivalents thereof, including at least one of the genes in Table 5; and

wherein the set comprises no more than 450 protein-coding genes.

A set of genes that “provides the information” required for replication of a free-living organism can be in any form that can be transcribed (e.g. into mRNA, rRNA or tRNA) and, in the case of protein-encoding sequences, translated into protein, wherein the transcription/translation products provide functions that allow the free-living organism to function.

This set of protein-coding genes is smaller than the complete complement of genes found in M genitalium (482 genes), the smallest known set of naturally occurring genes in a free-living organism.

A set of protein-coding genes of the invention can lack at least about 55 (e.g. at least about, 70, 80 or 90) of the genes listed in Table 2), and/or it can comprise at least about 360 (e.g. at least about 370 or 380) of the genes listed in Table 3.

A set of the invention can further comprise:

genes encoding an ABC transporter for phosphate import, selected from the group consisting of (a) MG410, MG411 and MG412, and (b) MG289, MG290 and MG291, and functional equivalents thereof; and/or

a lipoprotein-encoding gene selected from the group consisting of MG185 and MG260, and functional equivalents thereof; and/or

a glycerophosphoryl diester phosphodiesterase gene selected from the group consisting of MG293 and MG385, and functional equivalents thereof.

Furthermore, a set of the invention can further comprise the 43 RNA-coding genes of Mycoplasma genitalium , or functional equivalents thereof.

The genes in a set of the invention may constitute a chromosome; and/or may be from M. genitalium.

Another aspect of the invention is a free-living organism that can grow and replicate under axenic conditions in a rich bacterial culture medium (such as SP4), whose set of genes consists of a set of the invention, e.g. a set that comprises at least one gene involved in hydrogen or ethanol production.

Another aspect of the invention is a method for determining the function of a gene, comprising inserting, mutating or removing the gene into/in/from such a free-living organism, and measuring a property of the organism.

Another aspect of the invention is a method of hydrogen or ethanol production, comprising growing a free-living organism of that invention that comprises at least one gene involved in hydrogen or ethanol production, in a suitable medium such that hydrogen or ethanol is produced.

Another aspect of the invention is an effective subset of a set as noted above. An “effective subset,” as used herein, refers to a subset that provides the information required for replication of a free-living organism in a rich bacterial culture medium, such as SP4.

A minimal gene set of the invention has a variety of applications. For example, a minimal gene set of the invention can be introduced into cells of a microorganism, such as a bacterium, which lack a genome or a functional genome (e.g. ghost cells) and used experimentally to investigate requirements for cell growth, protein synthesis, replication or other bacterial functions under varying conditions. One or more of the minimal genes in the ghost cells can be modified or substituted with orthologous genes or genes or substituted with non-orthologous genes that express proteins which perform the same function(s), to allow structure/function studies of those genes. Cells comprising a minimal gene set of the invention can be modified to further comprise one or more expressible heterologous genes, either integrated into the genome or replicating on one or more independent plasmids. These cells can be used, e.g., to study properties or activities of the heterologous genes (e.g., structure/function studies), or to produce useful amounts of the heterologous proteins (e.g. biologic drugs, vaccines, catalytic enzymes, energy sources, etc).

As noted, a minimal gene set is one that provides the information required for replication of a free-living organism in a rich bacterial culture medium. The minimal gene set described herein was identified based on genes that were shown to be non-essential for bacterial growth in the medium SP4 (whose composition is described in reference #17), in the presence of tetracycline selection (the tetM tetracycline resistance gene is present in the transposon used to inactivate the genes which were shown to be non-essential). The set of non-essential genes may be different for organisms grown under different conditions (e.g. in different bacterial medium, under different selection conditions, etc). In general, a culture medium that supports growth and proliferation of a minimal organism (containing a gene set as discussed herein), with as few environmental stresses as possible, contains energy sources such as glucose, arginine or urea; protein or peptides; all amino acids; nucleotides; vitamins; cofactors; fatty acids and other membrane components such as cholesterol; enzyme cofactors; salts; minerals and buffers.

Such a medium is SP4 (Spiroplasma medium), which is a highly nutritious mixture of beef heart infusion, peptone supplemented with yeast extract, CMRL 1066 Medium and 17% fetal bovine serum. The yeast extract provides diphosphopyridine nucleotides and the serum provides cholesterol and a source of protein. (See, e.g., Tully et al. (1979) J. Infect. Dis 139, 478-82.) In particular, SP4 medium contains the following components:

Mix

| | | |
|---|---|---|
| Mycoplasma Broth Base | 3.5 | g |
| Bacto Tryptone | 10 | g |
| Bacto Peptone | 5.3 | g |
| Distilled water | 600 | ml |

Adjust pH to 7.5

Autoclave at 121° C. for 15 min

Add Aseptically

| | | |
|---|---|---|
| 20% Glucose | 25 | ml |
| CMRL 1066 (10X) | 50 | ml |
| 7.5% Sodium Bicarbonate | 14.6 | ml |
| 200 mM L-Glutamine | 5 | ml |
| Yeast extract Solution | 35 | ml |
| 2% Autoclaved TC Yeastolate | 100 | ml |
| Fetal Bovine Serum (Heat inactivated) | 170 | ml |
| Penicillin G (10⁷ IU/ml) | 100 | μl |

CMRL 1066 Components

| | 1X | |
|---|---|---|
| Chemical | Molarity (mM) | |
| Calcium chloride (CaCl2—2H2O) | 1.800 | |
| Potassium Chloride (KCl) | 5.300 | |
| Magnesium sulfate (MgSO4) | 0.814 | |
| Sodium chloride (NaCl) | 116.000 | |
| Sodium phosphate, mono (NaH2PO4) | 1.010 | |
| Thiamine pyrophosphate | 0.0021 | |
| Coenzyme A | 0.00326 | |
| 2′-deoxyadenosine | 0.0398 | |
| 2′-deoxycytidine | 0.4441 | |
| 2′-deoxyguanosine | 0.0375 | |
| Beta-nicotinamide adenine dinucleotide | 0.0105 | |
| Flavin adenine dinucleotide | 0.00127 | |
| D-Glucose | 3.33000 | |
| Glutathione reduced | 0.0325 | |
| 5-Methyl-2′-deoxycytidine | 0.0004 | |
| Phenol red | 0.0502 | |
| Sodium acetate-3H2O | 0.6100 | |
| d-Glucuronic acid | 0.0177 | |
| Thymidine | 0.0413 | |
| beta-nicotinamide adenine dinucleotide | 0.0013 | |
| phosphate | | |
| Tween 80 | 5 | mg/L |
| Uridine-5′-triphosphate | 0.0020 | |
| L-Alanine | 0.281 | |
| L-Arginine | 0.330 | |
| L-Aspartic acid | 0.230 | |
| L-Cystine | 1.480 | |
| L-Cysteine | 0.108 | |
| L-Glutamic | 0.510 | |
| Glycine | 0.667 | |
| L-Histidine | 0.952 | |
| trans-4-Hydroxy-L-proline | 0.763 | |
| L-Isoleucine | 0.153 | |
| L-Leucine | 0.458 | |
| L-Lysine | 0.383 | |
| L-Methionine | 0.101 | |
| L-Phenylalanine | 0.152 | |
| L-Proline | 0.348 | |
| L-Serine | 0.238 | |
| L-Threonine | 0.252 | |
| L-Tryptophan | 0.049 | |
| L-Tyrosine disodium salt | 0.260 | |
| L-Valine | 0.214 | |
| Biotin | 0.000041 | |
| D-Pantothenic acid hemicalcium salt | 0.000021 | |
| Choline Chloride | 0.0035 | |
| Folic acid | 0.0000227 | |
| myo-inositol | 0.0002 | |
| Niacinamide | 0.00203 | |
| Niacin | 0.0002 | |
| 4-Aminobenzoic Acid | 0.0003 | |
| Pyridoxal Hydrochloride | 0.0001 | |
| Pyridoxine Hydrochloride | 0.00012 | |
| Riboflavin | 0.0000266 | |
| Thiamine hydrochloride | 0.0000297 | |
| Ascorbic Acid | 0.284 | |
| Cholesterol | 0.000517 | |
| Sodium bicarbonate (NaHCO3) | 26.200 | |
| L-Glutamine | 2.000 | |

The term “gene,” as used herein, refers to a polynucleotide comprising a protein-coding or RNA-coding sequence, in an expressible form, e.g. operably linked to an expression control sequence. The “coding sequences” of the gene generally do not include expression control sequences, unless they are embedded within the coding sequence. In different embodiments of the invention, the coding sequences of the genes listed in Tables 2 to 5 can be under the control of the naturally occurring expression control sequences or they can be under the control of heterologous expression control sequences, or combinations thereof.

An “expression control sequence,” as used herein, refers to a polynucleotide sequence that regulates expression of a polypeptide coded for by a polynucleotide to which it is functionally (“operably”) linked. Expression can be regulated at the level of the mRNA or polypeptide. Thus, the term expression control sequence includes mRNA-related elements and protein-related elements. Such elements include promoters, domains within promoters, ribosome binding sequences, transcriptional terminators, etc. An expression control sequence is operably linked to a nucleotide sequence when the expression control sequence is positioned in such a manner to effect or achieve expression of the coding sequence. For example, when a promoter is operably linked 5′ to a coding sequence, expression of the coding sequence is driven by the promoter.

The minimal gene set suggested in the Examples herein is composed of genes or sequences from Mycoplasma genitalium (M. genitalium ) G37 (ATCC 33530). The complete genome of this bacterium is provided as Genbank accession number L43976. The individual genes are annotated in the Genbank listing as MG001, MG002 through MG470. The sequences of the genes were published on the TIGR web site in early October, 2005.

However, any of a variety of other protein- or RNA-coding genes or sequences can be substituted in a minimal gene set for the exemplified protein- or RNA-coding gene or sequences, provided that the protein or RNA encoded by the substituting gene can be expressed and that it provides a sufficient amount of the activity, function and/or structure to substitute for the M genitalium gene or sequence in a minimal gene set. Such substitutes are sometimes referred to herein as “functional equivalents” of the exemplified genes or coding sequences.

Suitable genes or coding sequences that can be substituted include, for example, an active mutant, variant, polymorph etc. of a M genitalium gene; or a corresponding (orthologous) gene from another bacterium, such as a different Mycoplasma species (e.g., M. capricolum). Furthermore, genes or sequences from the minimal gene set can be substituted with orthologous genes from an evolutionarily more diverse organism, such as an archaebacterium or a eukaryotic organism. Genes from eukaryotic organisms which must be post-translationally modified in order to function by a mechanism unavailable in a bacterial host cannot, of course, be used. Similarly, expression control sequences from eukaryotic genes can be used only if they can function in the background of a bacterial cell.

In one embodiment of the invention, genes from the minimal gene set are replaced by non-orthologous gene displacement (by a different set of genes providing an equivalent function or activity). For example, genes from the glycolytic pathway of M genitalium as shown in the Examples can be substituted with genes from a different organism that utilizes a different source for generating energy (such as hydrolysis of urea, fermentation of arginine, etc.).

For example, M genitalium generates energy via glycolysis. One can substitute a different energy generation system from another organism that would make most of the genes that express the enzymes of the glycolytic pathway superfluous. For instance energy generation in Ureaplasma parvum, a bacterium closely related to M genitalium is based on the hydrolysis of urea. That system includes 8 genes that encode the urease enzyme complex, two ammonium transporters, and as yet unidentified nickel ion transporter (presumably one of several U parvum cation transporters), and possibly a urea transporter (no transporter has been identified, and the very small urea molecule may enter the cell by diffusion). We expect that substitution of these 11-12 U parvum genes for 15-20 M genitalium genes encoding glycolytic enzymes and carbohydrate transporters would produce an organism with fewer genes capable more robust growth as is seen with U parvum.

As used herein, the term “polynucleotide” includes a single stranded DNA corresponding to the single strand provided in the Genbank listing, or to the complete complement thereto, or to the double stranded form of the molecule. Also included are RNA and DNA-like or RNA-like materials, such as branched DNAs, peptide nucleic acids (PNA) or locked nucleic acids (LNA).

Functional equivalents of genes can also include a variety of variant polynucleotides, provided that the variant polynucleotide can provide at least a measurable amount of the function of the original polynucleotide from which it varies. Preferably, the variant can provide at least about 50%, 75%, 90% or 95% of the function of the original polynucleotide. For example, a functional variant of a polynucleotide as described herein includes a polynucleotide that includes degenerate codons; or that is an active fragment of the original polynucleotide; or that exhibits at least about 90% identity (e.g. at least about 95% or 98% identity) with the original polynucleotide; or that can hybridize specifically to the original polynucleotide under conditions of high stringency.

Unless otherwise indicated, the term “about,” as used herein, refers to plus or minus 10%. Thus, about 90%, as used above, includes 81% to 99%. As used herein, the end points of a range are included with the range.

Functional variant polynucleotides may take a variety of forms, including, e.g., naturally or non-naturally occurring polymorphisms, including single nucleotide polymorphisms (SNPs), allelic variants, and mutants. They may comprise, e.g., one or more additions, insertions, deletions, substitutions, transitions, transversions, inversions, chromosomal translocations, variants resulting from alternative splicing events, or the like, or any combinations thereof.

The degree of sequence identity can be obtained by conventional algorithms, such as those described by Lipman and Pearson (Proc. Natl. Acad. Sci. 80:726-730, 1983) or Martinez/Needleman-Wunsch (Nucl Acid Research 11:4629-4634, 1983).

A polynucleotide that hybridizes specifically to a second polynucleotide under conditions of high stringency hybridizes preferentially to that polynucleotide. Conditions of “high stringency,” as used herein, means, for example, incubating a blot or other hybridization reaction overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity. Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

The minimal gene set suggested herein has been derived by taking into account some of the following factors. Furthermore, the minimal gene set may be modified, e.g. for growth under other culture conditions, taking into account some of the following factors:

Although the noted protein-coding genes appear to be essential for growth under the conditions of the experiments described herein, additional protein-coding genes may be required under other conditions. For example, we isolated mutants in DNA metabolism genes that were expendable for the duration of our experiment, but might be necessary for the long-term survival of the organism. These were six genes involved in recombination and DNA repair: recA (MG339), recU (MG352), Holliday junction DNA helicases ruvA (MG358) and ruvB (MG359), formamidopyrimidine-DNA glycosylase mutM (MG262.1), which excises oxidized purines from DNA, and a likely DNA damage inducible protein gene (MG360). Perhaps because of an accumulation of cell damage over time, mutants in chromosome segregation protein SMC (MG298) and hypothetical gene MG115, which is similar to the cinA gene of Streptococcus pneumoniae competence-inducible (cin) operon, grew more poorly after repeated passage.

Even with its near minimal gene set M genitalium has apparent enzymatic redundancy. We disrupted two complete ABC transporter gene cassettes for phosphate (MG410, MG411, MG412) and putatively phosphonate (MG289, MG290, MG291) import. The PhoU regulatory protein gene (MG409) was not disrupted, suggesting it is needed for both cassettes. Phosphate is an essential metabolite that must be imported. Either phosphate might be imported by both transporters as a result of relaxed substrate specificity by the phosphonate system, or there is a metabolic capacity to interconvert phosphate and phosphonate. Although we disrupted both of these three gene cassettes, cells presumably need at least one phosphate transporter. Therefore, a minimal gene set preferably contains three ABC transporter genes for phosphate importation. Relaxed substrate specificity is a recurring theme proposed and shown for several M genitalium enzymes as a mechanism by which this bacterium meets its metabolic needs with fewer genes (21, 22).

M genitalium generates ATP through glycolysis, and although none of the genes encoding enzymes involved in the initial glycolytic reactions were disrupted, mutations in two energy generation genes suggested there may be still more unexpected genomic redundancy in this essential pathway. We identified viable insertion mutants in genes encoding lactate/malate dehydrogenase (MG460) and the dihydrolipoamide dehydrogenase subunit of the pyruvate dehydrogenase complex (MG271). Mutations in either of these dehydrogenases would be expected to have glycolytic ATP production, and unbalanced NAD⁺ and NADH levels, which are the primary oxidizing and reducing agents in glycolysis. These mutations should have greatly reduced growth rate and accelerated acidification of the growth medium While the MG271 mutants grew about 20% slower than wild type cells, inexplicably, the lactate dehydrogenase mutants grow ˜20% faster. We also isolated a mutant in glycerol-3-phospate dehydrogenase (MG039), a phospholipid biosynthesis enzyme. The loss of functions in these mutants could have been compensated for by other M genitalium dehydrogenases or reductases. This could be another case of mycoplasma enzymes having a relaxed substrate specificity as has been reported for lactate/malate dehydrogenase (21) and nucleotide kinases (22).

Under our laboratory conditions we identified 101 non-essential protein-coding genes. It appears that the remaining 381 M genitalium protein-coding genes, plus three phosphate transporter genes, and 43 RNA-coding genes comprise the essential genes set for this minimal cell (Table 3). We disrupted genes in only 5 of the 12 M genitalium paralogous gene families. Only for the two families comprised of lipoproteins MG185 and MG260 and glycerophosphoryl diester phosphodiesterases MG293 and MG385 did we disrupt all members. Accordingly, these families' functions may be essential, and we expanded our projection of the essential gene set to 386 genes to include them (one each of MG185 or MG260, and one each of MG 293 and MG385). This is a significantly greater number of essential genes than the 265-350 predicted in the inventors' previous study of M genitalium (4), or in the gene knockout/disruption study that identified 279 essential genes in B. subtilis, which is a more conventional bacterium from the same Firmicutes taxon as M genitalium (6). Similarly, our finding of 386 essential protein-coding genes greatly exceeds theoretical projections of how many genes comprise a minimal genome such as Mushegian and Koonin's 256 genes shared by both H. influenzae and M genitalium (2), and the 206 gene core of a minimal bacterial gene set proposed by Gil et al (3). One of the surprises about the present essential gene set is its inclusion of 108 hypothetical proteins and proteins of unknown function.

These data suggest that a genome constructed to encode the 386 protein-coding and 43 structural RNA genes could sustain a viable synthetic cell, which has been referred to hypothetically as a Mycoplasma laboratorium (24). A variety of mechanisms can be used for preparing such a viable synthetic cell. For example, the minimal gene set can be introduced into a ghost cell, from which the resident genome has been removed or disabled. In one embodiment, ribosomes, membranes and other cellular components important for gene regulation, transcription, translation, post-transcriptional modification, secretion, uptake of nutrients or other substances, etc, are present in the ghost cell. In another embodiment, one or more of these components is prepared synthetically.

In one embodiment of the invention, the genes in the minimal gene set, or a subset of those genes, are cloned into conventional vectors, e.g. to form a library. The DNA to be cloned can be obtained from any suitable source, including naturally occurring genes, genes previously cloned into a different vector, or artificially synthesized genes. The genes may be cloned by in vitro, synthetic procedures, such as those disclosed in co-pending PCT application PCT/2006/16349, filed 1 May 2006, “Amplification and Cloning of Single DNA Molecules Using Rolling Circle Amplification,” incorporated by reference herein in its entirety. For example, synthetically prepared genes of the gene set may be amplified and assembled to form a synthetic gene or genome. This can be performed by diluting DNA molecules, such that each sample of diluted DNA contains, on average, one molecule of DNA, in fragments of about 5 kb, for example, and then converting to single stranded DNA circles, and then amplifying the DNA circles using Φ29 polymerase.

As a library, the gene sets of the invention can be arranged in any form, in single or multiple copies, and can be arranged in individual oligonucleotides each having a section of one of the genes, one of the genes, or more than one of the genes. These oligonucleotides can be arranged as cassettes. The cassettes can be joined up to form larger gene assemblies, including a minimal genome comprising or consisting of all the genes of the gene set of the invention. The genes can be assembled by a method such as that described in PCT International Patent Application No. PCT/US06/31214, filed 11 Aug. 2006, “Method For In Vitro Recombination Employing a 3′ Exonuclease Activity, ” incorporated by reference herein in its entirety. PCT/US06/31214 describes methods of joining cassettes of genes into larger assemblies, and can be used to produce a single DNA molecule comprising the gene set of the invention. In particular, that application describes an in vitro method, using isolated proteins, for joining two or more double-stranded (ds) DNA molecules of interest, wherein the distal region of the first DNA molecule and the proximal region of the second DNA molecule of each pair share a region of sequence identity, comprising (a) treating the DNA molecules with an enzyme having an exonuclease activity, under conditions effective to yield single-stranded overhanging portions of each DNA molecule which contain a sufficient length of the region of sequence homology to hybridize specifically to the region of sequence homology of its pair; (b) incubating the treated DNA molecules of (a) under conditions effective to achieve specific annealing of the single-stranded overhanging portions; and (c) treating the incubated DNA molecules in (b) under conditions effective to fill in remaining single-stranded gaps and to seal the nicks thus formed, wherein the region of sequence identity comprises at least 20 non-palindromic nucleotides (nt).

The DNA molecules of the library may have a size of any practical length. The lower size limit for a dsDNA to circularize is about 200 base pairs. Therefore, the total length of the joined fragments (including, in some cases, the length of the vector) is preferably at least about 200 bp in length. The DNAs can take the form of either a circle or a linear molecule. The library may include from two to a very large number of DNA molecules, which can be joined together. In general, at least about 10 fragments can be joined.

More particularly, the number of DNA molecules or cassettes that may be joined to produce an end product, in one or several assembly stages, may be at least or no greater than about 2, 3, 4, 6, 8, 10, 15, 20, 25, 50, 100, 200, 500, 1000, 5000, or 10,000 DNA molecules, for example in the range of about 4 to about 100 molecules. The DNA molecules or cassettes in a library of the invention may each have a starting size in a range of at least or no greater than about 80 bs, 100 bs, 500 bs, 1 kb, 3 kb, 5 kb, 6 kb, 10 kb, 18 kb, 20 kb, 25 kb, 32 kb, 50 kb, 65 kb, 75 kb, 150 kb, 300 kb, 500 kb, 600 kb, or larger, for example in the range of about 3 kb to about 100 kb. According to the invention, methods may be used for assembly of about 100 cassettes of about 6 kb each, into a DNA molecule of about 600 kb.

One embodiment of the invention is to join cassettes, such as 5-6 kb DNA molecules representing adjacent regions of a gene or genome included in a gene set of the invention, to create combinatorial assemblies. For example, it may be of interest to modify a bacterial genome, such as a putative minimal genome or a minimal genome, so that one or more of the genes is eliminated or mutated, and/or one or more additional genes is added. Such modifications can be carried out by dividing the genome into suitable cassettes, e.g. of about 5-6 kb, and assembling a modified genome by substituting a cassette containing the desired modification for the original cassette. Furthermore, if it is desirable to introduce a variety of changes simultaneously (e.g. a variety of modifications of a gene of interest, the addition of a variety of alternative genes, the elimination of one or more genes, etc.), one can assemble a large number of genomes simultaneously, using a variety of cassettes corresponding to the various modifications, in combinatorial assemblies. After the large number of modified sequences is assembled, preferably in a high throughput manner, the properties of each of the modified genomes can be tested to determine which modifications confer desirable properties on the genome (or an organism comprising the genome). This “mix and match” procedure produces a variety of test genomes or organisms whose properties can be compared. The entire procedure can be repeated as desired in a recursive fashion.

Methods of cloning, as well as many of the other molecular biological methods used in conjunction with the present invention, are discussed, e.g., in Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elseveir Sciences Publishing, Inc., New York; Hames et al. (1985), Nucleic Acid Hybridization, IL Press; Dracopoli et al. Current Protocols in Human Genetics, John Wiley & Sons, Inc.; and Coligan et al. Current Protocols in Protein Science, John Wiley & Sons, Inc.

Another aspect of the invention is a set of genes or polynucleotides on the invention which are in a free-living organism. The organism may be in a dormant or resting state (e.g., lyophilized, stored in a suitable solution, such as glycerol, or stored in culture medium), or it may growing and/or replicating, for example in a rich culture medium, such as SP4.

Another aspect of the invention is a set of polypeptides encoded by a set of genes or polynucleotides of the invention. The polypeptides may be, e.g., in a free-living organism.

Another aspect of the invention is a set of genes or polynucleotides of the invention that are recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable media can be used to create a manufacture comprising computer readable medium having recorded thereon a polynucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a set of nucleotide or amino acid sequences of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing a set of nucleotide or amino acid sequences of the invention in computer readable form, the skilled artisan can routinely access the sequence information for a variety of purposes. For example, one skilled in the art can use the nucleotide or amino acid sequences of the invention in computer readable form to compare the sequences with orthologous sequences that can be substituted for the present sequences in an alternative version of the minimal genome. Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

In the foregoing and in the following example, all temperatures are set forth in uncorrected degrees Celsius; and, unless otherwise indicated, all parts and percentages are by weight.

### EXAMPLES

I-Materials and Methods

A. Cells and plasmids. We obtained wild type M genitalium G37 (ATCC® Number: 33530™) from the American Type Culture Collection (Manassas, Va.). As part of this project we re-sequenced and re-annotated the genome of this bacterium. The new M genitalium G37 sequence (Genbank accession number CP000122) differed from the previous M genitalium (13) genome sequence at 34 sites. Several genes previously listed as having frameshifts were merged including MG 016, MG 017, and MG018 (DEAD helicase) and MG419 and MG420 (DNA polymerase III gamma/tau subunit). Our transposon mutagenesis vector was the plasmid pIVT-1, which contains the Tn4001 transposon with a tetracycline resistance gene (tetM)(15), and was a gift from Dr. Kevin Dybvig at the University of Alabama at Birmingham.

B. Transformation of M genitalium with Tn4001 by electroporation. Confluent flasks of M genitalium cells were harvested by scraping into electroporation buffer (EB) comprised of 8 mM HEPES+272 mM sucrose at pH 7.4. We washed and then resuspended the cells in a total volume of 200-300 μl EB. On ice, 100 μl cells were mixed with 30 μg pIVT-1 plasmid DNA and transferred to a 2 mm chilled electroporation cuvette (BioRad, Hercules, Calif.). We electroporated using 2500 V, 25 μF, and 100Ω. After electroporation we resuspended the cells in 1 ml of 37° C. SP4 medium and allowed the cells to recover for 2 hours at 37° C. with 5% CO₂. Aliquots of 200 μl of cells were spread onto SP4 agar plates containing 2 mg/l tetracycline hydrochloride (VWR, Bridgeport, N.J.). The plates were incubated for 3-4 weeks at 37° C. with 5% CO₂ until colonies were visible. When colonies were 3-4 weeks old, we transferred individual M genitalium colonies into SP4 medium +7 mg/L tetracycline in 96 well plates. We incubated the plates at 37° C. with 5% CO₂ until the SP4 in most of the wells began to turn acidic and became yellow or orange (˜4 days). We froze those mutant stock cells at −80° C.

C. Amplification of isolated colonies for DNA extraction. We inoculated 4 ml SP4 containing 7 μg/ml tetracycline in 6 well plates with 20 μl transposon mutant stock cells and incubated the plates at 37° C. with 5% CO₂ until the cells reached 100% confluence. To extract genomic DNA from confluent cells, we scraped the cells and then transferred the cell suspension to a tube for pelleting by centrifugation. Thus any non-adherent cells were not lost. We washed the cells in PBS (Mediatech, Herndon, Va.) and then resuspended them in a mixture of 100 μl PBS and 100 μl of the chaotropic MTL buffer from a Qiagen MagAttract DNA Mini M48 Kit (Qiagen, Valencia, Calif.). Tubes were stored at −20° C. until the genomic DNA could be extracted using a Qiagen BioRobot M48 workstation (Qiagen).

D. Location of Tn4001 tet insertion sites by DNA sequencing from M. genitalium genomic templates. Our 20 μl sequencing reactions contained ˜0.5 μg of genomic DNA, 6.4 pmol of the 30 base oligonucleotide GTACTCAATGAATTAGGTGGAAGACCGAGG (SEQ ID NO: 1) (Integrated DNA Technologies, Coralville, Iowa). The primer binds in the tetM gene 103 basepairs from one of the transposon/genome junctions. Using BLAST we located the insertion site on the M genitalium genome.

E. Quantitative PCR to determine colony homogeneity and genes duplication. We designed quantitative PCR primers (Integrated DNA Technologies) flanking transposon insertion sites using the default conditions for the primer design software Primer Express 1.5 (Applied Biosystems). Using quantitative PCR done on an Applied Biosystems 7700 Sequence Detection System, we determined the amounts of the target genes lacking a Tn4001 insertion in genomic DNA prepared from mutant colonies relative to a the amount of the those genes in wild type M genitalium . Reactions were done in Eurogentec qPCR Mastermix Plus SYBR Green (San Diego, Calif.). Genomic DNA concentrations were normalized after determining their relative amounts using a TaqMan quantitative PCR specific for the 16S rRNA gene that was done in Eurogentec qPCR Mastermix Plus. We calculated the amounts of target genes lacking the transposon in mutant genomic DNA preparations relative to the amounts in wild type using the delta-delta Ct method (16).

II. Identification of a Minimal Gene Set

We sequenced across the transposon-genome junctions of our mutants using a primer specific for Tn4001 tet. Presence of a transposon in the central region of a gene of a viable bacterium indicated that gene was disrupted and therefore non-essential (dispensable). We considered transposon insertions disruptive only if they were after the first three codons and before the 3′-most 20% of the coding sequence of a gene. Thus, non-disruptive mutations resulting from transposon mediated duplication of short sequences at the insertion site (18, 19), and potentially inconsequential COOH-terminal insertions do not result in erroneous determination of gene expendability. Without wishing to be bound by any particular theory, it is suggested that these disruptions actually occurred, even though theoretically, some genes might tolerate transposon insertions, and we did not confirm the absence of the gene products. To exclude the possibility that gene disruptions were the result of a transposon insertion in one copy of a duplicated gene, we used PCR to detect genes lacking the insertion. This showed us that almost all of our colonies contained both disrupted and wild type versions of the genes identified as having the Tn4001. Further analysis using quantitative PCR showed most colonies were mixtures of two or more mutants, thus we operationally refer to them and any DNA isolated from them as colonies rather than clones. This cell clumping led us to isolate individual mutants using filter cloning. To do this we forced cells through 0.22 μm filters before plating to break up clumps of cells possibly containing multiple different mutants. We used these cells to produce subcolonies which we both sequenced and analyzed using quantitative PCR. For each disrupted gene we subcloned at least one primary colony.

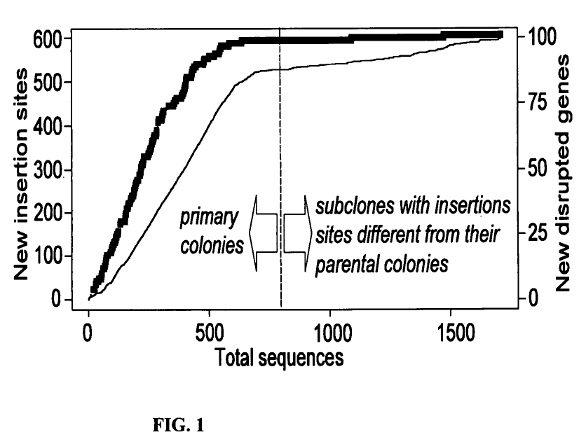

In total we analyzed 3,152 M genitalium transposon insertion mutant primary colonies, and subcolonies to determine the locations of Tn4001 tet inserts. For 75% of these we generated sequence data that enabled us to map the transposon insertion sites. Colonies containing multiple Tn4001 tet insertions cannot be characterized using this approach. Only 62% of primary colonies generated useful sequence. This was likely because of the tendency of mycoplasma cells to form persistent cell aggregates leading to colonies containing mixtures of multiple mutants that proved refractory to sequencing. For subcolonies the success rate was 82%. Of the successfully sequenced subcolonies in 59% the transposon insert was at a different site than in the parental primary colony. The rate at which we identified mutants with previously unhit insertion sites on the genome was higher for the primary colonies than the subcolonies. However the rate of accumulation of new insertion sites dropped after our first 600 colonies, indicating we were approaching saturation mutagenesis of all non-lethal insertion sites (FIG. 1).

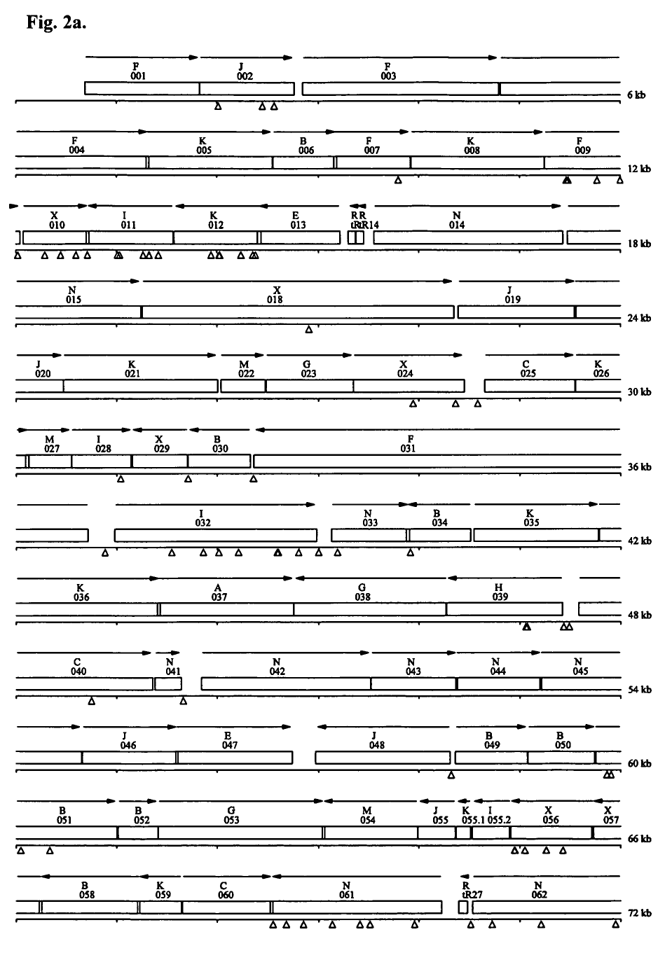

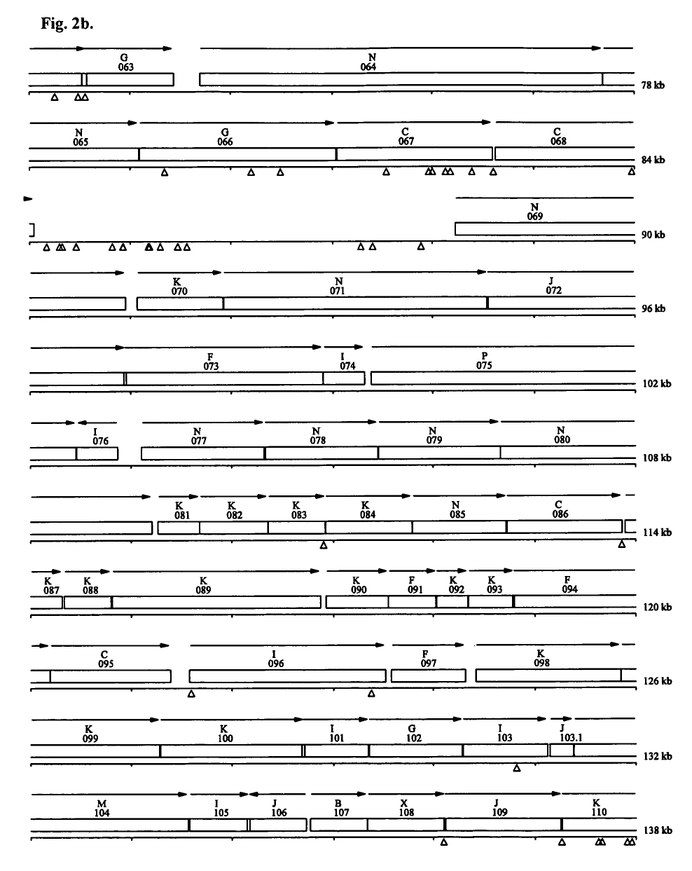

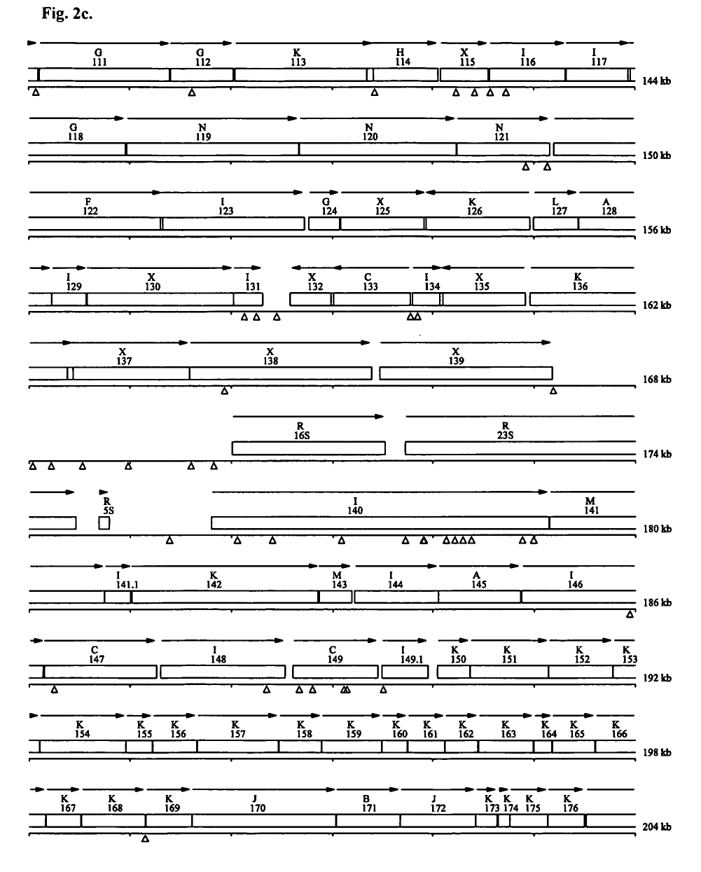

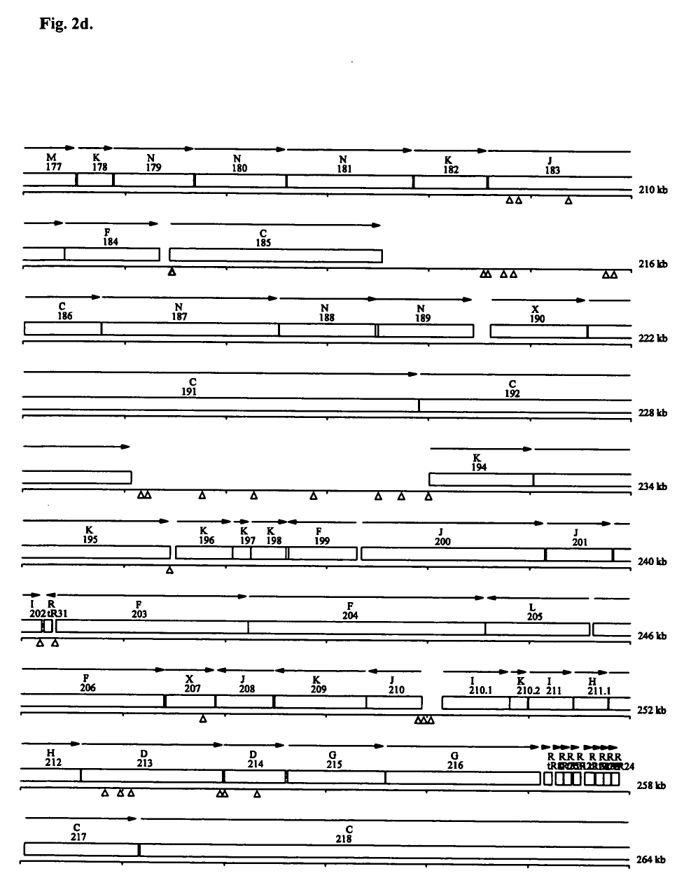

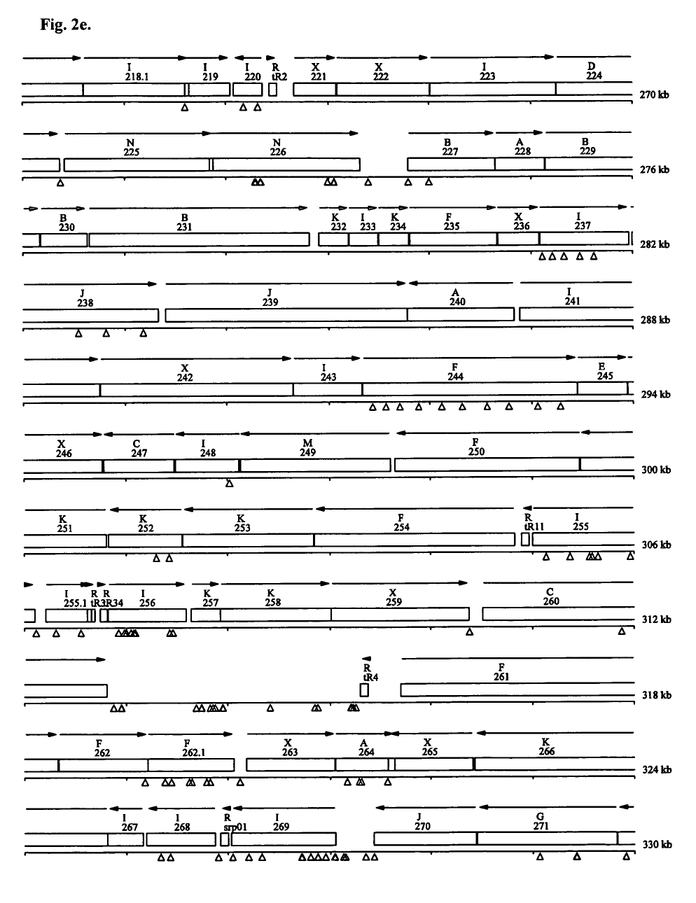

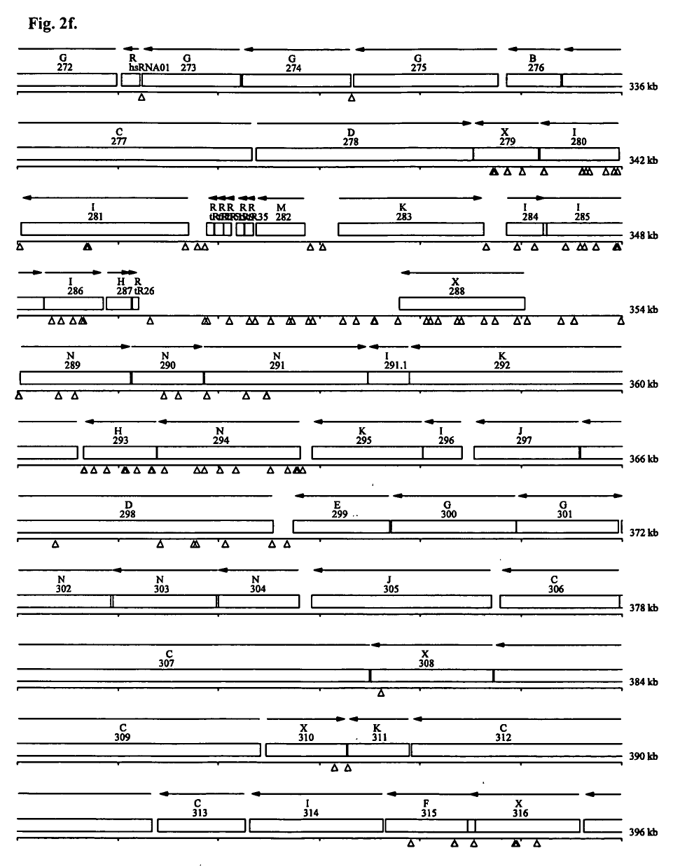

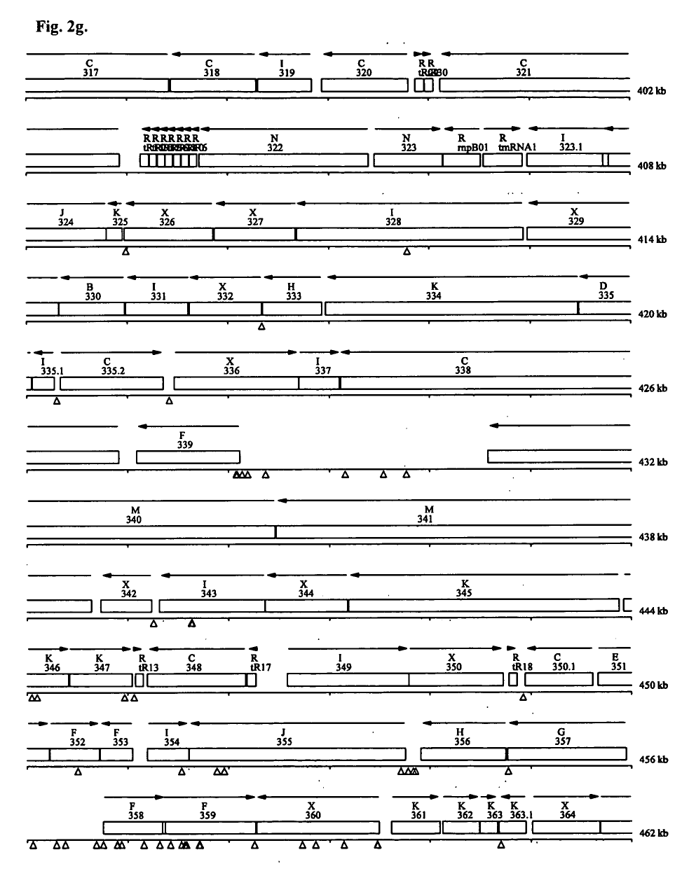

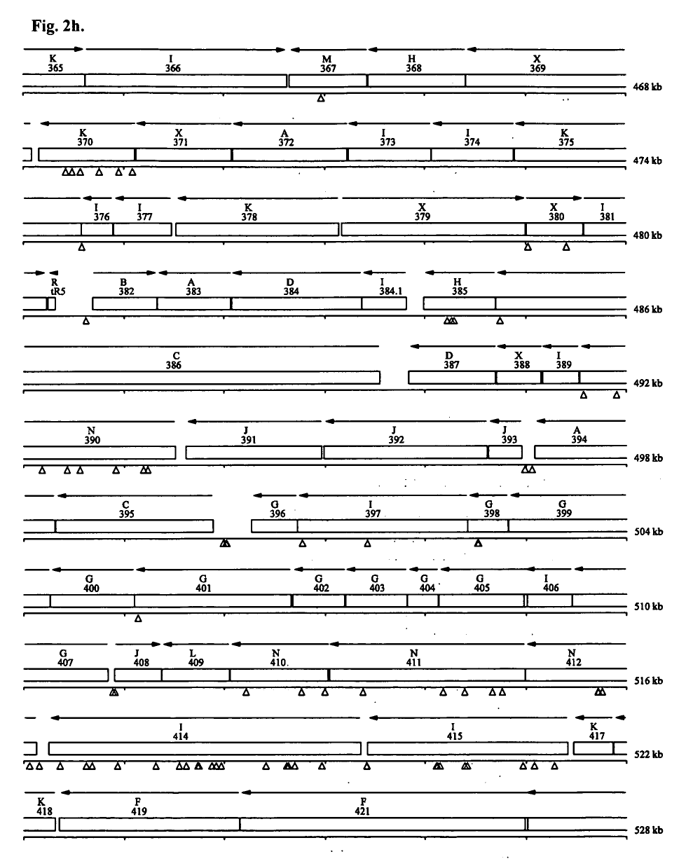

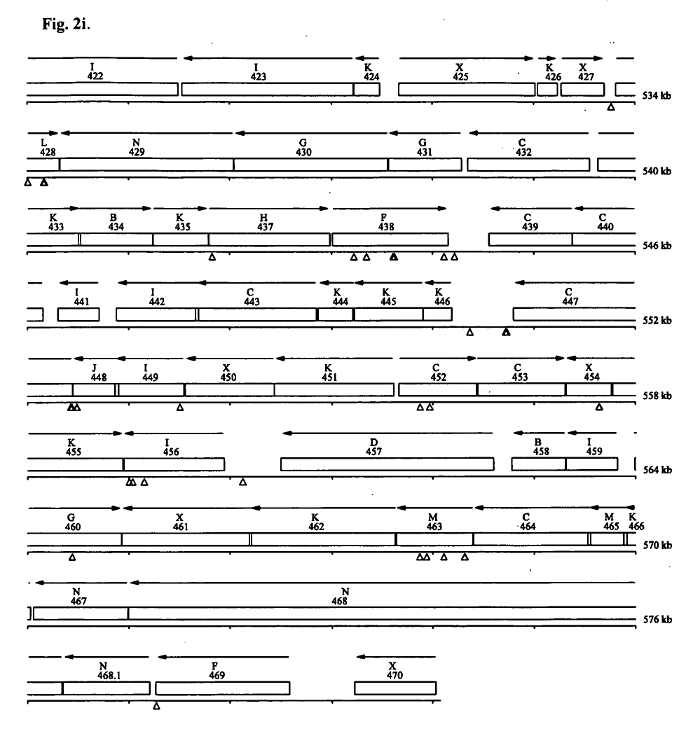

We mapped a total of 2293 different transposon insertion sites on the genome (FIG. 2). Eighty-seven percent of the mutations were in protein-coding genes. None of the 43 RNA encoding genes (for rRNA, tRNA, or structural RNA) contained insertions. To address the question of which M genitalium genes were not essential for growth in SP4 (17), a rich laboratory medium, we used the following criteria to designate a gene disruption. We considered transposon insertions disruptive if they were after the first three codons and before the 3′-most 20% of the coding sequence of a gene. Thus, non-disruptive mutations resulting from transposon mediated duplication of short sequences at the insertion site (18, 19), and potentially inconsequential COOH-terminal insertions do not result in erroneous determination of gene expendability. Using these criteria we identified a total of 101 dispensable M genitalium genes (Table 2). In FIG. 1, it can be seen that new genes disrupted as a function of primary colonies and subcolonies plateaus, suggesting that we have or very nearly have disrupted all non-essential genes. Transposon mutants in non-essential genes were able to form colonies on solid agar, and isolated colonies were able to grow in liquid culture, both under tetracycline selection.

We wanted to determine if any of our disrupted genes were in cells bearing two copies of the gene. Unexpectedly, PCRs using primers flanking the transposon insertion sites produced amplicons of the size expected for wild type templates from all 5 colonies initially tested. End-stage analysis of PCRs could not tell us if the wild type sequences we amplified were the result of a low level of transposon jumping out of the target gene, or if there was a gene duplication. To address this, for at least one colony or subcolony for each disrupted gene we used quantitative PCR to measure how many copies of contaminating wild type versions of that gene there were in the sequenced DNA preps.

Analysis of the quantitative PCR results showed most colonies were mixtures of multiple mutants. This was likely a consequence of our high transformation efficiency and the tendency of mycoplasma cells to aggregate. The direct genomic sequencing identified only the plurality member of the population. To address this issue we adapted our mutant isolation protocol to include one or two rounds of filter cloning. Existing colonies of interest were filter subcloned. We isolated 10 subcolonies and the sites of their Tn4001 insertions were determined. We took both rapidly growing colonies and M genitalium colonies that were delayed in their appearance. Often only a minority of the subcolonies had inserts in the same location as found with the parental colony. After filter cloning we still found that almost every subcolony had some low level of a wild type copy of the disrupted gene. This is likely the result of Tn4001 jumping (20). After subcloning we were able to isolate gene disruption mutant colonies for 100 of our 101 different disrupted M genitalium genes that had less than 1% wild type sequence.

Several mutants manifested remarkable phenotypes. While many of the mutants grew slowly, mutants in lactate/malate dehydrogenase (MG460), and conserved hypothetical proteins MG414 and MG415 mutants had doubling times up to 20% faster than wild type M genitalium (data not shown). Cells with transposon insertions in the transketolase gene (MG066), which encodes a membrane protein and pentose phosphate pathway enzyme, grew in chains of clumped cells rather than in the monolayers characteristic of wild type M genitalium . Other mutant cells grew in suspension rather than adhering to plastic. Some cells would lyse when washed with PBS, and thus had to be processed in either SP4 medium or 100% serum.

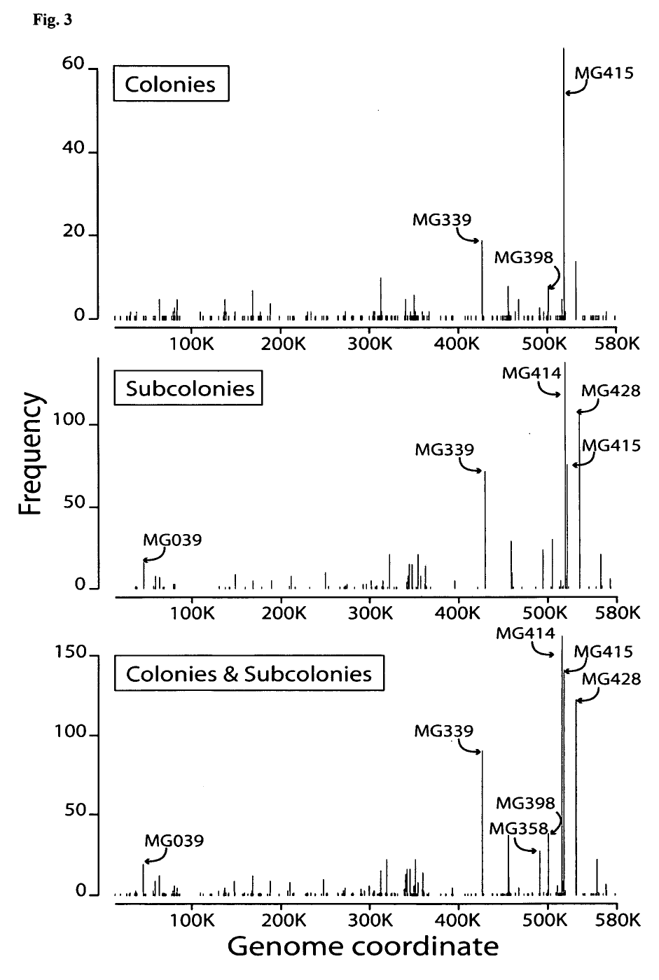

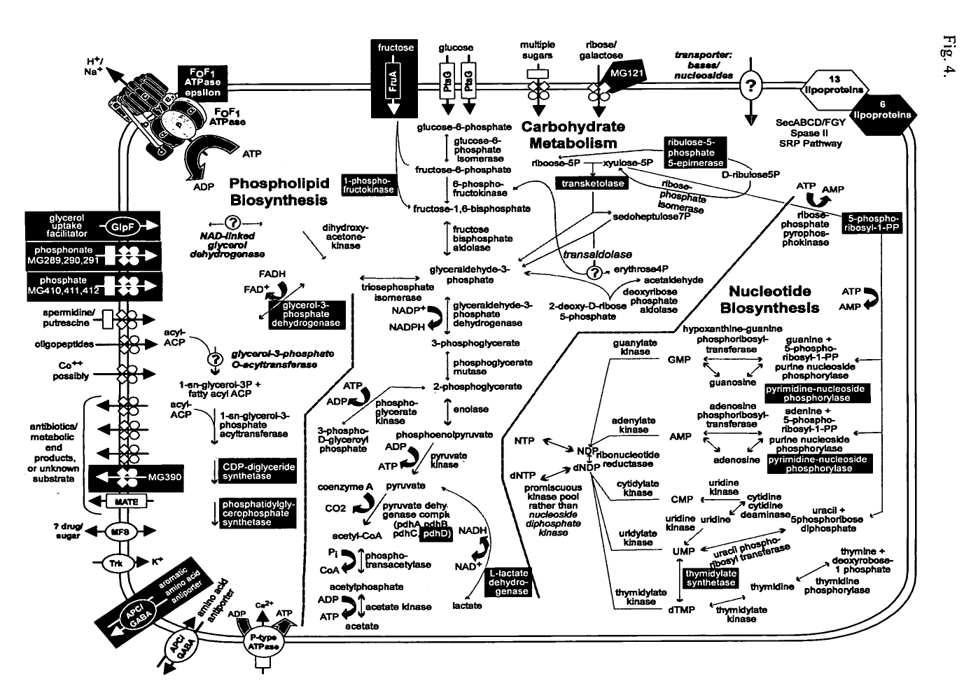

We isolated mutants with transposon insertions at some sites much more frequently than others (FIG. 3). We found colonies with mutations at hot spots in four genes: MG339 (recA), the fast growing MG414 and MG415 and MG428 (putative regulatory protein) comprised 31% of the total mutant pool. There was a striking difference in the most frequently found transposon insertion sites among primary colonies relative to the subcolonies having different insertions sites than their parental colonies (FIG. 3). We isolated 169 colonies and subcolonies having different insertion sites than their parental colonies with Tn4001 tet inserted at basepair 517, 751, which is in MG414. Only 5 (3%) of those were primary colonies. Conversely, we isolated 209 colonies with inserts in the 520,114 to 520,123 region, which is in MG415, and 56% of those were in primary colonies. The MG414 mutants were probably due both to rapid growth and to Tn4001 preferential jumping to that genome region, whereas the high frequency and near equal distribution of MG415 primary and subcolony transposon insertions may only be because those mutants grow more rapidly than others.

III. Verification (or Modification) of the Minimal Gene Set

As noted above, at least 386 protein-coding genes and all of the RNA genes are essential and could form a minimal set. However, it seems unlikely that all of those “one-at-a time” dispensable genes could be eliminated simultaneously. To determine a subset that can be simultaneously deleted, a wild type chromosome is constructed synthetically. The synthetic genome is constructed hierarchically from chemically synthesized oligonucleotides. Subsets of the dispensable genes are then removed. The synthetic natural chromosome and the reduced genome are tested for viability by transplantation into cells from which the resident chromosome has been removed. Rapid advances in gene synthesis technology and efforts at developing genome transplantation methods allow the confirmation that the M genitalium essential gene set described above is a true minimal gene set, or provide a basis to modify that gene set.

### REFERENCES

1. Ferber, D. (2004) Science 303, 158-61.
2. Mushegian, A. R. & Koonin, E. V. (1996) Proc Natl Acad Sci USA 93, 10268-73.
3. Gil, R., Silva, F. J., Pereto, J. & Moya, A. (2004) Microbiol Mol Biol Rev 68, 518-37, table of contents.
4. Hutchison, C. A., Peterson, S. N., Gill, S. R., Cline, R. T., White, O., Fraser, C. M., Smith, H. O. & Venter, J. C. (1999) Science 286, 2165-9.
5. Forsyth, R. A., Haselbeck, R. J., Ohlsen, K. L., Yamamoto, R. T., Xu, H., Trawick, J. D., Wall, D., Wang, L., Brown-Driver, V., Froelich, J. M. & et al. (2002) Mol Microbiol 43, 1387-400.
6. Kobayashi, K., Ehrlich, S. D., Albertini, A., Amati, G., Andersen, K. K., Arnaud, M., Asai, K., Ashikaga, S., Aymerich, S., Bessieres, P. & et al. (2003) Proc Natl Acad Sci USA 100, 4678-83.
7. Salama, N. R., Shepherd, B. & Falkow, S. (2004) J Bacteriol 186, 7926-35.
8. Herring, C. D., Glasner, J. D. & Blattner, F. R. (2003) Gene 311, 153-63.
9. Mori, H., Isono, K., Horiuchi, T. & Miki, T. (2000) Res Microbiol 151, 121-8.
10. Ji, Y., Zhang, B., Van, S. F., Horn, Warren, P., Woodnutt, G., Burnham, M. K. & Rosenberg, M. (2001) Science 293, 2266-9.
11. Reich, K. A., Chovan, L. & Hessler, P. (1999) J Bacteriol 181, 4961-8.
12. Sassetti, C. M., Boyd, D. H. & Rubin, E. J. (2001) Proc Natl Acad Sci USA 98, 12712-7.
13. Fraser, C. M., Gocayne, J. D., White, O., Adams, M. D., Clayton, R. A., Fleischmann, R. D., Bult, C. J., Kerlavage, A. R., Sutton, G., Kelley, J. M. & et al. (1995) Science 270, 397-403.
15. Dybvig, K., French, C. T. & Voelker, L. L. (2000) J Bacteriol 182, 4343-7.
15a. Pour-El, I., Adams, C. and Minion, F. C. (2002). Plasmid 47, 129-37.
16. Relative Quantitation of Gene Expression (1997) The Perkin-Elmer Corporation., Foster City, Calif.
17. Tully, J. G., Rose, D. L., Whitcomb, R. F. & Wenzel, R. P. (1979) J Infect Dis 139, 478-82.
18. Dyke, K. G., Aubert, S. & el Solh, N. (1992) Plasmid 28, 235-46.
19. Rice, L. B., Carias, L. L. & Marshall, S. H. (1995) Antimicrob Agents Chemother 39, 1147-53.
20. Mahairas, G. G., Lyon, B. R., Skurray, R. A. & Pattee, P. A. (1989) J Bacteriol 171, 3968-72.
21. Cordwell, S. J., Basseal, D. J., Pollack, J. D. & Humphery-Smith, I. (1997) Gene 195, 113-20.
22. Pollack, J. D., Myers, M. A., Dandekar, T. & Herrmann, R. (2002) Omics 6, 247-58.
23. Dhandayuthapani, S., Rasmussen, W. G. & Baseman, J. B. (1999) Proc Natl Acad Sci USA 96, 5227-32.
24. Reich, K. A. (2000) Res Microbiol 151, 319-24.

Tables:

TABLE 1
Paralogous gene families in bacteria used for gene essentiality studies.

| | | Genes in | | | Fraction of | |
|---|---|---|---|---|---|---|
| | Protein | paralogous | | Average | genes in | Maximum |
| | coding | gene | Paralogous | family | paralogous | family |
| Species | genes | families | families | size | gene families | size |
| Mycoplasma genitalium | 483 M. genitalium | 29 | 12 | 2.4 | 6.0% | 4 |
| Bacillus subtilis | 4106 | 1221 | 421 | 2.9 | 29.7% | 55 |
| Escherichia coli (K-12) | 4254 | 1287 | 432 | 3.0 | 30.3% | 52 |
| Haemophilus influenzae | 1709 | 190 | 73 | 2.6 | 11.1% | 26 |
| Helicobacter pylori | 1566 | 192 | 71 | 2.7 | 12.3% | 13 |
| Mycobacterium bovis | 3953 | 1294 | 336 | 3.9 | 32.7% | 146 |
| Pseudomonas | 5566 | 2247 | 593 | 3.8 | 40.4% | 114 |
| aeruginosa | | | | | | |
| Staphylococcus aureus | 2714 | 628 | 225 | 2.8 | 23.1% | 44 |

We used a common definition for members of paralogous gene families requiring they have 30% identity over 60% of the length of the longer protein sequence (a single linkage clustering then defines the families).

TABLE 2
Mycoplasma genitalium genes with Tn4001tet insertions that are disrupted. Genes are
grouped by functional roles.

| | | | | | |
|---|---|---|---|---|---|
| Locus | Symbol | Common name | A | B | C |

Biosynthesis of cofactors, prosthetic groups, and carriers

| | | | |
|---|---|---|---|
| MG264 | dephospho-CoA kinase | x | x |

Cell envelope

| | | |
|---|---|---|
| MG040 | lipoprotein, putative | |
| MG067 | lipoprotein, putative | x |
| MG147 | Lipoprotein, putative | |
| MG149 | lipoprotein, putative | |
| MG185 | lipoprotein, putative | |
| MG260 | lipoprotein, putative | |

Cellular processes

| | | | |
|---|---|---|---|
| MG238 | tig | trigger factor | x |

DNA metabolism

| | | | | | |
|---|---|---|---|---|---|
| MG009 | | deoxyribonuclease, TatD family, putative | | x | x |
| MG213 | scpA | segregation and condensation protein A | | | |
| MG214 | | segregation and condensation protein B | x | | |
| MG244 | | UvrD/REP helicase | x | x | |
| MG262.1 | mutM | formamidopyrimidine-DNA glycosylase | | x | |
| MG298 | smc | chromosome segregation protein SMC | x | x | |
| MG315 | | DNA polymerase III, delta subunit, putative | | x | x |
| MG339 | recA | recA protein (recombinase A) | | x | |
| MG352 | recU | recombination protein U | | | |
| MG358 | ruvA | Holliday junction DNA helicase | | x | |
| MG359 | ruvB | Holliday junction DNA helicase RuvB | | x | |
| MG438 | | type I restriction modification DNA specificity domain protein | | x | |

Energy metabolism

| | | | | | |
|---|---|---|---|---|---|
| MG063 | fruK | 1-phosphofructokinase, putative | x | x | |
| MG066 | tkt | transketolase | x | x | x |
| MG112 | rpe | ribulose-phosphate 3-epimerase | | x | x |
| MG271 | lpdA | dihydrolipoamide dehydrogenase | | x | |
| MG398 | atpC | ATP synthase F1, epsilon subunit | | x | x |
| MG460 | ldh | L-lactate dehydrogenase/malate dehydrogenase | | x | x |

Fatty acid and phospholipid metabolism

| | | | | |
|---|---|---|---|---|
| MG039 | | FAD-dependent glycerol-3-phosphate dehydrogenase, putative | x | |
| MG293 | | glycerophosphoryl diester phosphodiesterase family protein | x | |
| MG385 | | glycerophosphoryl diester phosphodiesterase family protein | x | |
| MG437 | cdsA | phosphatidate cytidylyltransferase | x | x |

Hypothetical proteins

| | | |
|---|---|---|
| MG011 | conserved hypothetical protein | x |
| MG032 | conserved hypothetical protein | |
| MG096 | conserved hypothetical protein | |
| MG103 | conserved hypothetical protein | |
| MG116 | conserved hypothetical protein | |
| MG131 | conserved hypothetical protein, authentic framehsift | |
| MG134 | conserved hypothetical protein | |
| MG140 | conserved hypothetical protein | x |
| MG149.1 | conserved hypothetical protein | |
| MG220 | conserved hypothetical protein | |
| MG237 | conserved hypothetical protein | |
| MG248 | conserved hypothetical protein | |
| MG255 | conserved hypothetical protein | |
| MG255.1 | conserved hypothetical protein | |
| MG256 | conserved hypothetical protein | |
| MG268 | conserved hypothetical protein | x |
| MG269 | conserved hypothetical protein | |
| MG280 | conserved hypothetical protein | |
| MG281 | conserved hypothetical protein | |
| MG284 | conserved hypothetical protein | |
| MG285 | conserved hypothetical protein | |
| MG286 | conserved hypothetical protein | |
| MG328 | conserved hypothetical protein | |
| MG343 | conserved hypothetical protein | |
| MG397 | conserved hypothetical protein | |
| MG414 | conserved hypothetical protein | |
| MG415 | conserved hypothetical protein | |
| MG449 | conserved hypothetical protein, authentic frameshift | x |
| MG456 | conserved hypothetical protein | |

Protein fate

| | | | |
|---|---|---|---|
| MG002 | | DnaJ domain protein | x |
| MG183 | | oligoendopeptidase F | x |
| MG210 | | signal peptidase II | x |
| MG238 | tig | trigger factor | x |
| MG355 | clpB | ATP-dependent Clp protease, ATPase subunit | x |
| MG408 | msrA | methionine-S-sulfoxide reductase | x |

Protein synthesis

| | | | | | |
|---|---|---|---|---|---|
| MG012 | | alpha-L-glutamate ligases, RimK family, putative | | x | |
| MG110 | rsgA | ribosome small subunit-dependent GTPase A | | | |
| MG252 | | RNA methyltransferase, TrmH family, group 3 | | x | |
| MG346 | | RNA methyltransferase, TrmH family, group 2 | x | x | x |
| MG370 | | pseudouridine synthase, RluA family | | x | |
| MG463 | | dimethyladenosine transferase | | x | x |

Purines, pyrimidines, nucleosides, and nucleotides

| | | | | |
|---|---|---|---|---|
| MG051 | pdp | pyrimidine-nucleoside phosphorylase | x | |
| MG227 | thyA | thymidylate synthase | x | x |

Regulatory functions

| | |
|---|---|
| MG428 | LuxR bacterial regulatory protein, putative |

Transcription

| | | | | | |
|---|---|---|---|---|---|
| MG367 | rnc | ribonuclease III | x | x | x |

Transport and binding proteins

| | | | | |
|---|---|---|---|---|
| MG033 | glpF | glycerol uptake facilitator | x | |
| MG061 | | Mycoplasma MFS transporter | | x |
| MG062 | fruA | PTS system, fructose-specific IIABC component | | x |
| MG121 | | ABC transporter, permease protein | | x |
| MG226 | | amino acid-polyamine-organocation (APC) permease family protein | | x |
| MG289 | | phosphonate ABC transporter, substrate binding protein (P37), | | |
| | | putative | | |
| MG290 | | phosphonate ABC transporter, ATP-binding protein, putative | | |
| MG291 | | phosphonate ABC transporter, permease protein (P69), putative | | |
| MG294 | | major facilitator superfamily protein, putative | | x |
| MG390 | | ABC transporter, ATP-binding/permease protein | | |
| MG410 | pstB | phosphate ABC transporter, ATP-binding protein | | x |
| MG411 | | phosphate ABC transporter, permease protein PstA | | x |
| MG412 | | phosphate ABC transporter, substrate-binding protein | | |

Unknown function

| | | | | |
|---|---|---|---|---|
| MG010 | | DNA primase-related protein | x | |
| MG018 | | helicase SNF2 family, putative | x | |
| M0024 | ychF | GTP-binding protein YchF | x | x |
| MG056 | | tetrapyrrole (corrin/porphyrin) methylase protein | x | x |
| MG115 | | competence/damage-inducible protein CinA domain protein | | |
| MG138 | lepA | GTP-binding protein LepA | x | x |
| MG207 | | Ser/Thr protein phosphatase family protein | | |
| MG279 | | expressed protein of unknown function | | |
| MG316 | | ComEC/Rec2-related protein | x | |
| MG360 | | ImpB/MucB/SamB family protein | x | |
| MG380 | | methyltransferase GidB | X | |
| MG454 | | OsmC-like protein | | |

All information is based on the M genitalium genome sequence and annotation reported herein. Genes are grouped by main biological roles. The columns are as follows:

M genitalium gene locus

Gene symbol

Gene common name
A. Orthologous genes essential in Bacillus. subtilis(1).
B. In theoretical minimal 256 gene set defined by Mushegian and Koonin as orthologous genes present in M genitalium and H. influenzae(2).
C. In theoretical 206 gene core of a minimal genome set defined by Gil et al (3).

### REFERENCES

1 Kobayashi, K., Ehrlich, S. D., Albertini, A., Amati, G., Andersen, K. K., Arnaud, M., Asai, K., Ashikaga, S., Aymerich, S., Bessieres, P., et al. (2003) Proc Natl Acad Sci US A 100, 4678-83.
2. Mushegian, A. R. & Koonin, E. V. (1996) Proc Natl Acad Sci USA 93, 10268-73.

3. Gil, R., Silva, F. J., Pereto, J. & Moya, A. (2004) Microbiol Mol Biol Rev 68, 518-37, table of contents.

TABLE 3
Mycoplasma genitalium protein coding genes that were not disrupted in this study.
Genes are grouped by functional roles.

| | | | | | |
|---|---|---|---|---|---|
| Locus | Symbol | Common name | A | B | C |

Biosynthesis of cofactors, prosthetic groups, and carriers

| | | | | | |
|---|---|---|---|---|---|
| MG037 | | nicotinate phosphoribosyltransferase (NAPRTase) family | | x | x |
| MG128 | | inorganic polyphosphate/ATP-NAD kinase, probable | x | x | |
| MG145 | ribF | riboflavin biosynthesis protein RibF | | x | x |
| MG228 | dhfR | dihydrofolate reductase | x | x | x |
| MG240 | | nicotinamide-nucleotide adenylyltransferase/conserved hypothetical | x | | |
| | | protein | | | |
| MG383 | | NH(3)-dependent NAD+ synthetase, putative | x | x | |
| MG394 | glyA | serine hydroxymethyltransferase | x | x | x |

Cell envelope

| | | | |
|---|---|---|---|
| MG025 | | glycosyl transferase, group 2 family protein | x |
| MG060 | | glycosyl transferase, group 2 family protein | x |
| MG068 | | lipoprotein, putative | x |
| MG095 | | lipoprotein, putative | |
| MG133 | | membrane protein, putative | |
| MG191 | mgpA | MgPa adhesin | x |
| MG192 | p110 | P110 protein | x |
| MG217 | | proline-rich P65 protein | |
| MG218 | hmw2 | HMW2 cytadherence accessory protein | |
| MG247 | | membrane protein, putative | x |
| MG277 | | membrane protein, putative | |
| MG306 | | membrane protein, putative | |
| MG307 | | lipoprotein, putative | |
| MG309 | | lipoprotein, putative | |
| MG312 | hmw1 | HMW1 cytadherence accessory protein | x |
| MG313 | | membrane protein, putative | x |
| MG317 | hmw3 | HMW3 cytadherence accessory protein | x |
| MG318 | p32 | P32 adhesin | x |
| MG320 | | membrane protein, putative | |
| MG321 | | lipoprotein, putative | |
| MG335.2 | | glycosyl transferase, group 2 family protein | |
| MG338 | | lipoprotein, putative | |
| MG348 | | lipoprotein, putative | |
| MG350.1 | | membrane protein, putative | |
| MG386 | p200 | P200 protein | x |
| MG395 | | lipoprotein, putative | |
| MG432 | | membrane protein, putative | |
| MG439 | | lipoprotein, putative | |
| MG440 | | lipoprotein, putative | |
| MG443 | | membrane protein, putative | |
| MG447 | | membrane protein, putative | |
| MG453 | galU | UTP-glucose-1-phosphate uridylyltransferase | x |
| MG464 | | membrane protein, putative | |

Cell/organism defense

| | |
|---|---|
| MG075 | 116 kDa surface antigen |

Cellular processes

| | | | | | |
|---|---|---|---|---|---|
| MG224 | ftsZ | cell division protein FtsZ | x | x | x |
| MG278 | relA | GTP pyrophosphokinase | | x | |
| MG335 | | GTP-binding protein engB, putative | | x | |
| MG384 | obg | GTPase1 Obg | | x | x |
| MG387 | era | GTP-binding protein Era | | x | x |
| MG457 | ftsH | ATP-dependent metalloprotease FtsH | | x | x |

Central intermediary metabolism

| | | | | | |
|---|---|---|---|---|---|
| MG013 | folD | methylenetetrahydrofolate dehydrogenase/methylenetetrahydrofolate | x | x | |
| | | cyclohydrolase | | | |
| MG047 | metK | S-adenosylmethionine synthetase | x | x | x |
| MG245 | | 5-formyltetrahydrofolate cyclo-ligase, putative | | x | |
| MG351 | ppa | inorganic pyrophosphatase | | x | x |

DNA metabolism

| | | | | | |
|---|---|---|---|---|---|
| MG001 | dnaN | DNA polymerase III, beta subunit | x | x | x |
| MG003 | gyrB | DNA gyrase, B subunit | x | x | x |
| MG004 | gyrA | DNA gyrase, A subunit | x | x | x |
| MG007 | | DNA polymerase III delta prime subunit, putative | x | x | x |
| MG031 | polC | DNA polymerase III, alpha subunit, Gram-positive type | x | x | x |
| MG073 | uvrB | excinuclease ABC, B subunit | | x | |
| MG091 | | single-strand binding protein family | x | x | x |
| MG094 | dnaB | replicative DNA helicase | x | x | x |
| MG097 | | uracil-DNA glycosylase, putative | | x | x |
| MG122 | topA | DNA topoisomerase I | x | x | |
| MG184 | | adenine-specific DNA modification methylase | | x | |
| MG186 | | Staphylococcal nuclease homologue, putative | | | |
| MG199 | rnhC | ribonuclease HIII | | | |
| MG203 | parE | DNA topoisomerase IV, B subunit | x | x | |
| MG204 | parC | DNA topoisomerase IV, A subunit | x | x | |
| MG206 | | excinuclease ABC, C subunit | | x | |
| MG235 | | apurinic endonuclease (APN1) | | x | x |
| MG250 | | DNA primase | x | x | x |
| MG254 | ligA | DNA ligase, NAD-dependent | x | x | x |
| MG261 | polC-2 | DNA polymerase III, alpha subunit | | x | x |
| MG262 | | 5′-3′ exonuclease, putative | | x | x |
| MG353 | | DNA-binding protein HU, putative | | x | x |
| MG419 | | DNA polymerase III, subunit gamma and tau | | | |
| MG421 | uvrA | excinuclease ABC, A subunit | | x | |
| MG469 | | chromosomal replication initiator protein DnaA | x | x | |

Energy metabolism

| | | | | | |
|---|---|---|---|---|---|
| MG023 | fba | fructose-1,6-bisphosphate aldolase, class II | x | x | x |
| MG038 | glpK | glycerol kinase | | x | |
| MG050 | deoC | deoxyribose-phosphate aldolase | | x | |
| MG053 | | phosphoglucomutase/phosphomannomutase, putative | | x | |
| MG102 | trxB | thioredoxin-disulfide reductase | x | x | x |
| MG111 | pgi | glucose-6-phosphate isomerase | | x | x |
| MG118 | galE | UDP-glucose 4-epimerase | | x | |
| MG124 | trx | thioredoxin | x | | x |
| MG215 | pfk | 6-phosphofructokinase | x | x | x |
| MG216 | pyk | pyruvate kinase | | x | x |
| MG272 | pdhC | dihydrolipoamide acetyltransferase | | x | |
| MG273 | pdhB | pyruvate dehydrogenase component E1, beta subunit | | x | |
| MG274 | pdhA | pyruvate dehydrogenase component E1, alpha subunit | | x | |
| MG275 | nox | NADH oxidase | | x | |
| MG299 | pta | phosphate acetyltransferase | | x | |
| MG300 | pgk | phosphoglycerate kinase | x | x | x |
| MG301 | gap | glyceraldehyde-3-phosphate dehydrogenase, type I | | x | x |
| MG357 | ackA | acetate kinase | | x | |
| MG396 | rpiB | ribose 5-phosphate isomerase B | | x | x |
| MG399 | atpD | ATP synthase F1, beta subunit | | x | x |
| MG400 | atpG | ATP synthase F1, gamma subunit | | x | x |
| MG401 | atpA | ATP synthase F1, alpha subunit | | x | x |
| MG402 | atpH | ATP synthase F1, delta subunit | | x | x |
| MG403 | atpF | ATP synthase F0, B subunit | | x | x |
| MG404 | atpE | ATP synthase F0, C subunit | | x | x |
| M0405 | atpB | ATP synthase F0, A subunit | | x | x |
| MG407 | eno | enolase | x | x | x |
| MG430 | gpmI | 2,3-bisphosphoglycerate-independent phosphoglycerate mutase | x | x | x |
| MG431 | tpiA | triosephosphate isomerase | x | x | x |

Fatty acid and phospholipid metabolism

| | | | | | |
|---|---|---|---|---|---|
| MG114 | | CDP-diacylglycerol--glycerol-3-phosphate 3-phosphatidyltransferase | x | x | |
| MG211.1 | acpS | holo-(acyl-carrier-protein) synthase | x | | |
| MG212 | | 1-acyl-sn-glycerol-3-phosphate acyltransferase, putative | | x | x |
| MG287 | | acyl carrier protein, putative | x | x | |
| MG333 | | acyl carrier protein phosphodiesterase, putative | x | x | |
| MG356 | | choline/ethanolamine kinase, putative | | | |
| MG368 | plsX | fatty acid/phospholipid synthesis protein PlsX | | x | |

Hypothetical proteins

| | | | |
|---|---|---|---|
| MG028 | conserved hypothetical protein | | |
| MG055.2 | conserved hypothetical protein | | |
| MG074 | conserved hypothetical protein | | |
| MG076 | conserved hypothetical protein | | |
| MG101 | conserved hypothetical protein | | |
| MG105 | conserved hypothetical protein | | |
| MG117 | conserved hypothetical protein | | |
| MG123 | conserved hypothetical protein | | |
| MG129 | conserved hypothetical protein | | |
| MG141.1 | conserved hypothetical protein | | |
| MG144 | conserved hypothetical protein | | |
| MG146 | conserved hypothetical protein | x | x |
| MG148 | conserved hypothetical protein | | |
| MG202 | conserved hypothetical protein | | |
| MG210.1 | conserved hypothetical protein | | |
| MG211 | conserved hypothetical protein | | |
| MG218.1 | conserved hypothetical protein | | |
| MG219 | Hypothetical protein | | |
| MG223 | conserved hypothetical protein | | |
| MG233 | conserved hypothetical protein | | |
| MG241 | conserved hypothetical protein | | |
| MG243 | conserved hypothetical protein | | |
| MG267 | conserved hypothetical protein | | |
| MG291.1 | conserved hypothetical protein | | x |
| MG296 | conserved hypothetical protein | | |
| MG314 | conserved hypothetical protein | x | |
| MG319 | conserved hypothetical protein | | |
| MG323.1 | conserved hypothetical protein | | |
| MG331 | conserved hypothetical protein | | |
| MG335.1 | conserved hypothetical protein | | |
| MG337 | conserved hypothetical protein | | |
| MG349 | conserved hypothetical protein | | |
| MG354 | conserved hypothetical protein | | |
| MG366 | conserved hypothetical protein | | |
| MG373 | conserved hypothetical protein | | |
| MG374 | conserved hypothetical protein | | |
| MG376 | conserved hypothetical protein | | |
| MG377 | conserved hypothetical protein | | |
| MG381 | conserved hypothetical protein | | |
| MG384.1 | conserved hypothetical protein | | |
| MG389 | conserved hypothetical protein | | |
| MG406 | conserved hypothetical protein | x | |
| MG422 | conserved hypothetical protein | | |
| MG423 | conserved hypothetical protein | x | |
| MG441 | conserved hypothetical protein | | |
| MG442 | GTP-binding conserved hypothetical protein | | |
| MG459 | conserved hypothetical protein | | |

Protein fate

| | | | | | |
|---|---|---|---|---|---|
| MG019 | dnaJ | chaperone protein DnaJ | | x | x |
| MG020 | pip | proline iminopeptidase | | x | |
| MG046 | | metalloendopeptidase, putative, glycoprotease family | | x | x |
| MG048 | ffh | signal recognition particle protein | x | x | x |
| MG055 | | preprotein translocase, SecE subunit | x | | x |
| MG072 | secA | preprotein translocase, SecA subunit | x | x | x |
| MG086 | | prolipoprotein diacylglyceryl transferase | | x | |
| MG103.1 | | preprotein translocase, SecG subunit | | | |
| MG106 | def | peptide deformylase | | x | |
| MG109 | | serine/threonine protein kinase, putative | | x | |
| MG170 | secY | preprotein translocase, SecY subunit | x | x | x |
| MG172 | map | methionine aminopeptidase, type I | x | x | x |
| MG200 | | DnaJ domain protein | | x | |
| MG201 | | co-chaperone GrpE | | x | x |
| MG208 | | glycoprotease family protein | | | |
| MG239 | lon | ATP-dependent protease La | | x | x |
| MG270 | | lipoyltransferase/lipoate-protein ligase, putative | | x | |
| MG297 | ftsY | signal recognition particle-docking protein FtsY | x | x | x |
| MG305 | dnaK | chaperone protein DnaK | | x | x |
| MG324 | | metallopeptidase family M24 aminopeptidase | | x | |
| MG391 | | cytosol aminopeptidase | | x | x |
| MG392 | groL | chaperonin GroEL | x | x | x |
| MG393 | groES | chaperonin, 10 kDa (GroES) | x | x | x |
| MG448 | msrB | methionine-R-sulfoxide reductase | | x | |

Protein synthesis

| | | | | | |
|---|---|---|---|---|---|
| MG005 | serS | seryl-tRNA synthetase | x | x | x |
| MG008 | | tRNA modification GTPase TrmE | | x | x |
| MG021 | metG | methionyl-tRNA synthetase | x | x | x |
| MG026 | efp | translation elongation factor P | | x | x |
| MG035 | hisS | histidyl-tRNA synthetase | x | x | x |
| MG036 | aspS | aspartyl-tRNA synthetase | x | x | x |
| MG055.1 | rpmG-2 | ribosomal protein L33 type 2 | | | |
| MG059 | smpB | SsrA-binding protein | | x | x |
| MG070 | rpsB | ribosomal protein S2 | x | x | x |
| MG081 | rplk | ribosomal protein L11 | x | x | x |
| MG082 | rplA | ribosomal protein L1 | x | x | x |
| MG083 | pth | peptidyl-tRNA hydrolase | x | x | x |
| MG084 | | tRNA(Ile)-lysidine synthetase | | | x |
| MG087 | rpsL | ribosomal protein S12 | x | x | x |
| MG088 | rpsG | ribosomal protein S7 | x | x | x |
| MG089 | fusA | translation elongation factor G | x | x | x |
| MG090 | | ribosomal protein S6 | x | x | x |
| MG092 | rpsR | ribosomal protein S18 | x | x | x |
| MG093 | | ribosomal protein L9 | x | x | x |
| MG098 | | glutamyl-tRNA(Gln) and/or aspartyl-tRNA(Asn) amidotransferase, C | x | | |
| | | subunit | | | |
| MG099 | | glutamyl-tRNA(Gln) and/or aspartyl-tRNA(Asn) amidotransferase, A | x | x | |
| | | subunit | | | |
| MG100 | gatB | glutamyl-tRNA(Gln) and/or aspartyl-tRNA(Asn) amidotransferase, B | x | | x |
| | | subunit | | | |
| MG113 | asnS | asparaginyl-tRNA synthetase | x | x | x |
| MG126 | trpS | tryptophanyl-tRNA synthetase | x | x | x |
| MG136 | lysS | lysyl-tRNA synthetase | x | x | x |
| MG142 | infB | translation initiation factor IF-2 | x | x | x |
| MG150 | rpsJ | ribosomal protein S10 | x | x | x |
| MG151 | rplC | ribosomal protein L3 | x | x | x |
| MG152 | rplD | ribosomal protein L4/L1 family | x | x | x |
| MG153 | rplW | ribosomal protein L23 | x | x | x |
| MG154 | rplB | ribosomal protein L2 | x | x | x |
| MG155 | rpsS | ribosomal protein S19 | x | x | x |
| MG156 | rplV | ribosomal protein L22 | x | x | x |
| MG157 | rpsC | ribosomal protein S3 | x | x | x |
| MG158 | rplP | ribosomal protein L16 | x | x | x |
| MG159 | rpmC | ribosomal protein L29 | x | x | x |
| MG160 | rpsQ | ribosomal protein S17 | x | x | x |
| MG161 | rplN | ribosomal protein L14 | x | x | x |
| MG162 | rplX | ribosomal protein L24 | x | x | x |
| MG163 | rplE | ribosomal protein L5 | x | x | x |
| MG164 | rpsN | ribosomal protein S14 | x | x | x |
| MG165 | rpsH | ribosomal protein S8 | x | x | x |
| MG166 | rplF | ribosomal protein L6 | x | x | x |
| MG167 | rplR | ribosomal protein L18 | x | x | x |
| MG168 | rpsE | ribosomal protein S5 | x | x | x |
| MG169 | rplO | ribosomal protein L15 | x | x | x |
| MG173 | infA | translation initiation factor IF-1 | x | x | x |
| MG174 | rpmJ | ribosomal protein L36 | x | x | x |
| MG175 | rpsM | ribosomal protein S13 | x | x | x |
| MG176 | rpsK | ribosomal protein S11 | x | x | x |
| MG178 | rplQ | ribosomal protein L17 | x | x | x |
| MG182 | | tRNA pseudouridine synthase A | | x | |
| MG194 | pheS | phenylalanyl-tRNA synthetase, alpha subunit | x | x | x |
| MG195 | | phenylalanyl-tRNA synthetase, beta subunit | x | x | x |
| MG196 | infC | translation initiation factor IF-3 | x | x | x |
| MG197 | rpmI | ribosomal protein L35 | x | x | x |
| MG198 | rplT | ribosomal protein L20 | x | x | x |
| MG209 | | pseudouridine synthase, RluA family | | x | |
| MG210.2 | rpsU | ribosomal protein S21 | | | |
| MG232 | rplU | ribosomal protein L21 | x | x | x |
| MG234 | rpmA | ribosomal protein L27 | x | x | x |
| MG251 | glyS | glycyl-tRNA synthetase | x | x | x |
| MG253 | cysS | cysteinyl-tRNA synthetase | x | x | x |
| MG257 | rpmE | ribosomal protein L31 | x | x | x |
| MG258 | prfA | peptide chain release factor 1 | x | x | x |
| MG266 | leuS | leucyl-tRNA synthetase | x | x | x |
| MG283 | proS | prolyl-tRNA synthetase | x | x | x |
| MG292 | alaS | alanyl-tRNA synthetase | x | x | x |
| MG295 | trmU | tRNA (5-methylaminomethyl-2-thiouridylate)-methyltransferase | x | x | x |
| MG311 | rpsD | ribosomal protein S4 | x | | x |
| MG325 | rpmG | ribosomal protein L33 | x | x | x |
| MG334 | valS | valyl-tRNA synthetase | x | x | x |
| MG345 | ileS | isoleucyl-tRNA synthetase | x | x | x |
| MG347 | | tRNA (guanine-N(7)-)-methyltransferase | | x | |
| MG361 | | ribosomal protein L10 | x | x | x |
| MG362 | rplL | ribosomal protein L7/L12 | x | x | x |
| MG363 | rpmF | ribosomal protein L32 | x | x | x |
| MG363.1 | | ribosomal protein S20 | x | x | x |
| MG365 | | methionyl-tRNA formyltransferase | x | x | |
| MG372 | | thiamine biosynthesis/tRNA modification protein ThiI | | | |
| MG375 | thrS | threonyl-tRNA synthetase | | x | x |
| MG378 | argS | arginyl-tRNA synthetase | x | x | x |
| MG417 | rpsI | ribosomal protein S9 | x | x | x |
| MG418 | rplM | ribosomal protein L13 | x | x | x |
| MG424 | rpsO | ribosomal protein S15 | x | x | x |
| MG426 | rpmB | ribosomal protein L28 | x | x | x |
| MG433 | tsf | translation elongation factor Ts | x | x | x |
| MG435 | frr | ribosome recycling factor | x | x | x |
| MG444 | rpls | ribosomal protein L19 | x | x | x |
| MG445 | trmD | tRNA (guanine-N1)-methyltransferase | x | x | |
| MG446 | rpsP | ribosomal protein S16 | x | x | x |
| MG451 | tuf | translation elongation factor Tu | x | x | x |
| MG455 | tyrS | tyrosyl-tRNA synthetase | x | x | x |
| MG462 | gltX | glutamyl-tRNA synthetase | x | x | x |
| MG466 | rpL34 | ribosomal protein L34 | x | x | x |

Purines, pyrimidines, nucleosides, and nucleotides

| | | | | | |
|---|---|---|---|---|---|
| MG006 | tmk | thymidylate kinase | x | x | x |
| MG030 | upp | uracil phosphoribosyltransferase | | x | x |
| MG034 | tdk | thymidine kinase | | x | |
| MG049 | deoD | purine nucleoside phosphorylase | | x | |
| MG052 | | cytidine deaminase | | x | |
| MG058 | prs | ribose-phosphate pyrophosphokinase | | x | x |
| MG107 | gmk | guanylate kinase | x | x | x |
| MG171 | adk | adenylate kinase | x | x | x |
| MG229 | nrdF | ribonucleoside-diphosphate reductase, beta chain | x | x | x |
| MG230 | nrdI | nrdI protein | x | | |
| MG231 | nrdE | ribonucleoside-diphosphate reductase, alpha chain | x | x | x |
| MG276 | apt | adenine phosphoribosyltransferase | | x | |
| MG330 | cmk | cytidylate kinase | x | x | |
| MG382 | udk | uridine kinase | | x | |
| MG434 | pyrH | uridylate kinase | | x | |
| MG458 | hpt | hypoxanthine phosphoribosyltransferase | x | x | x |

Regulatory functions

| | | |
|---|---|---|
| MG127 | Spx subfamily protein | x |
| MG205 | heat-inducible transcription repressor HrcA, putative | |

Transcription

| | | | | | |
|---|---|---|---|---|---|
| MG022 | | DNA-directed RNA polymerase, delta subunit | | x | |
| MG027 | nusB | transcription termination/antitermination protein NusB | | | |
| MG054 | | transcription antitermination protein NusG, putative | | x | x |
| MG104 | | ribonuclease R | | x | |
| MG141 | nusA | transcription termination factor NusA | x | x | x |
| MG143 | rbfA | ribosome-binding factor A | | x | x |
| MG177 | rpoA | DNA-directed RNA polymerase, alpha subunit | x | x | x |
| MG249 | rpoD | RNA polymerase sigma factor RpoD | | x | x |
| MG282 | greA | transcription elongation factor GreA | | x | x |
| MG340 | rpoC | DNA-directed RNA polymerase, beta′ subunit | x | x | x |
| MG341 | rpoB | DNA-directed RNA polymerase, beta subunit | x | x | x |
| MG465 | rnpA | ribonuclease P protein component | x | x | x |

Transport and binding proteins

| | | | | |
|---|---|---|---|---|
| MG014 | | ABC transporter, ATP-binding/permease protein | x | |
| MG015 | | ABC transporter, ATP-binding/permease protein | x | |
| MG041 | | phosphocarrier protein HPr | x | x |
| MG042 | | spermidine/putrescine ABC transporter, ATP-binding protein, | x | |
| | | putative | | |
| MG043 | | spermidine/putrescine ABC transporter, permease protein, putative | x | |
| MG044 | | spermidine/putrescine ABC transporter, permease protein, putative | x | |
| MG045 | | ABC transporter, spermidine/putrescine binding protein, putative | x | |
| MG064 | | ABC transporter, permease protein, putative | | |
| MG065 | | ABC transporter, ATP-binding protein | x | |
| MG069 | ptsG | PTS system, glucose-specific IIABC component | x | x |
| MG071 | | ATPase, P-type (transporting), HAD superfamily, subfamily IC | x | |
| MG077 | | oligopeptide ABC transporter, permease protein (OppB) | x | |
| MG078 | | oligopeptide ABC transporter, permease protein (OppC) | x | |
| MG079 | oppD | oligopeptide ABC transporter, ATP-binding protein | x | |
| MG080 | oppF | oligopeptide ABC transporter, ATP-binding protein | x | |
| MG085 | hprK | HPr(Ser) kinase/phosphatase | | |
| MG119 | | ABC transporter, ATP-binding protein | x | |
| MG120 | | ABC transporter, permease protein | x | |
| MG179 | | metal ion ABC transporter, ATP-binding protein, putative | | |
| MG180 | | metal ion ABC transporter ATP-binding protein, putative | x | |
| MG181 | | metal ion ABC transporter, permease protein | | |
| MG187 | | ABC transporter, ATP-binding protein | x | |
| MG188 | | ABC transporter, permease protein | x | |
| MG189 | | ABC transporter, permease protein | x | |
| MG225 | | amino acid-polyamine-organocation (APC) permease family protein | x | |
| MG302 | | metal ion ABC transporter, permease protein, putative | x | |
| MG303 | | metal ion ABC transporter, ATP-binding protein, putative | x | |
| MG304 | | metal ion ABC transporter, ATP binding protein, putative | | |
| MG322 | | potassium uptake protein, TrkH family, putative | x | |
| MG323 | | potassium uptake protein, TrkA family | x | |
| MG409 | | phosphate transport system regulatory protein PhoU, putative | x | |
| MG429 | ptsI | phosphoenolpyruvate-protein phosphotransferase | x | x |
| MG467 | | ABC transporter, ATP-binding protein | x | |
| MG468 | | ABC transporter, permease protein | | |
| MG468.1 | | ABC transporter, ATP-binding protein | | |

Unknown function

| | | | | | |
|---|---|---|---|---|---|
| MG029 | | DJ-1/PfpI family protein | | | |
| MG057 | | small primase-like protein | | | |
| MG108 | | protein phosphatase 2C, putative | | x | |
| MG125 | | Cof-like hydrolase, putative | | x | |
| MG130 | | uncharacterized domain HDIG | | | |
| MG132 | | HIT domain protein | | | x |
| MG137 | | UDP-galactopyranose mutase | | | |
| MG139 | | metallo-beta-lactamase superfamily protein | | x | |
| MG190 | | phosphoesterase, DHH subfamily 1 | | x | |
| MG221 | mraZ | mraZ protein | | x | |
| MG222 | | S-adenosyl-methyltransferase MraW | | x | x |
| MG236 | | expressed protein of unknown function | | | |
| MG242 | | expressed protein of unknown function | | | |
| MG246 | | Ser/Thr protein phosphatase family protein | | | |
| MG259 | | modification methylase, HemK family | | x | x |
| MG263 | | Cof-like hydrolase | | | |
| MG265 | | Cof-like hydrolase | | x | |
| MG288 | | expressed protein of unknown function | | | |
| MG308 | | ATP-dependent RNA helicase, DEAD/DEAH box family | | x | |
| MG310 | | hydrolase, alpha/beta fold family | | | |
| MG326 | | degV family protein | | x | |
| MG327 | | hydrolase, alpha/beta fold family | | | |
| MG329 | engA | GTP-binding protein engA | | | x |
| MG332 | | expressed protein of unknown function | | x | |
| MG336 | | aminotransferase, class V | x | x | x |
| MG342 | | NADPH-dependent FMN reductase domain protein | | | |
| MG344 | | hydrolase, alpha/beta fold family | | x | |
| MG350 | | expressed protein of unknown function | | | |
| MG364 | | expressed protein of unknown function | | | |
| MG369 | | DAK2 phosphatase domain protein | | | |
| MG371 | | DHH family protein | | | |
| MG379 | gidA | glucose-inhibited division protein A | | x | x |
| MG388 | | expressed protein of unknown function | | | x |
| MG425 | | ATP-dependent RNA helicase, DEAD/DEAH box family | | x | x |
| MG427 | | OsmC-like protein | | | |
| MG450 | | degV family protein | | | |
| MG461 | | HD domain protein | | x | |
| MG470 | | CobQ/CobB/MinD/ParA nucleotide binding domain | | x | |

| RNA Gene Name | 5′ End | 3′ End |
|---|---|---|
| tRNA-Ala-1 | 15369 | 15294 |
| tRNA-Ile-1 | 15451 | 15375 |
| tRNA-Ser-1 | 70481 | 70393 |
| Mg16SA | 171525 | |
| Mg23SA | 174465 | |
| Mg5SA | 174793 | |
| tRNA-Thr-1 | 240286 | 240213 |
| tRNA-Cys-1 | 257158 | 257234 |
| tRNA-Pro-1 | 257269 | 257345 |
| tRNA-Met-1 | 257349 | 257425 |
| tRNA-Met-2 | 257445 | 257521 |
| tRNA-Ser-2 | 257559 | 257650 |
| tRNA-Met-3 | 257664 | 257740 |
| tRNA-Asp-1 | 257742 | 257815 |
| tRNA-Phe-1 | 257818 | 257893 |
| tRNA-Arg-1 | 266423 | 266499 |
| tRNA-Gly-1 | 304965 | 304892 |
| tRNA-Arg-2 | 306617 | 306691 |
| tRNA-Trp-1 | 306740 | 306813 |
| tRNA-Arg-3 | 315377 | 315301 |
| Mg_srp01 | 326006 | 325924 |
| Mg_hsRNA01 | 331215 | 331034 |
| tRNA-Gly-2 | 343957 | 343884 |
| tRNA-Leu-1 | 344050 | 343965 |
| tRNA-Lys-1 | 344125 | 344051 |
| tRNA-Gln-1 | 344246 | 344172 |
| tRNA-Tyr-1 | 344337 | 344251 |
| tRNA-SeC-1 | 349128 | 349202 |
| tRNA-Ser-3 | 399868 | 399958 |
| tRNA-Ser-4 | 399960 | 400048 |
| tRNA-Leu-2 | 403218 | 403134 |
| tRNA-Lys-2 | 403299 | 403224 |
| tRNA-Thr-2 | 403381 | 403306 |
| tRNA-Val-1 | 403458 | 403383 |
| tRNA-Thr-3 | 403541 | 403467 |
| tRNA-Glu-1 | 403620 | 403544 |
| tRNA-Asn-1 | 403701 | 403627 |
| Mg_rnpB01 | 406519 | 406142 |
| MgtmRNA1 | 406542 | 406929 |
| tRNA-His-1 | 445078 | 445153 |
| tRNA-Leu-3 | 446265 | 446178 |
| tRNA-Leu-4 | 448783 | 448864 |
| tRNA-Arg-4 | 480315 | 480240 |

All information is based on the M genitalium genome sequence and annotation reported herein. Genes are grouped by main biological roles. The columns for the protein coding genes are as follows:

M genitalium gene locus

Gene symbol

Gene common name
A. Orthologous genes essential in Bacillus. subtilis(1).
B. In theoretical minimal 256 gene set defined by Mushegian and Koonin as orthologous genes present in M genitalium and H. influenzae(2).
C. In theoretical 206 gene core of a minimal genome set defined by Gil et al (3).

### REFERENCES

1 Kobayashi, K., Ehrlich, S. D., Albertini, A., Amati, G., Andersen, K. K., Arnaud, M., Asai, K., Ashikaga, S., Aymerich, S., Bessieres, P., et al. (2003) Proc Natl Acad Sci USA 100, 4678-83.
2. Mushegian, A. R. & Koonin, E. V. (1996) Proc Natl Acad Sci USA 93, 10268-73.

3. Gil, R., Silva, F. J., Pereto, J. & Moya, A. (2004) Microbiol Mol Biol Rev 68, 518-37, table of contents.

TABLE 4
Mycoplasma genitalium genes with Tn4001tet insertions that were not reported as being
disrupted (dispensable) in the 1999 study by Hutchison et al., but which have been shown to be
dispensable in the present study. Genes are grouped by functional roles.

| | | | | | |
|---|---|---|---|---|---|
| | Gene | | | | |
| Locus | Symbol | Common Name | A | B | C |

Cell envelope

| | |
|---|---|
| MG147 | membrane protein, putative (disrupted 7/06 using different tn40001 |
| | system) |

DNA metabolism

| | | | | | |
|---|---|---|---|---|---|
| MG214 | | segregation and condensation protein B | x | | |
| MG262.1 | mutM | formamidopyrimidine-DNA glycosylase | | x | |
| MG298 | smc | chromosome segregation protein SMC | x | x | |
| MG315 | | DNA polymerase III, delta subunit, putative | | x | x |
| MG358 | ruvA | Holliday junction DNA helicase | | x | |
| MG359 | ruvB | Holliday junction DNA helicase RuvB | | x | |

Energy metabolism

| | | | | | |
|---|---|---|---|---|---|
| MG063 | fruK | 1-phosphofructokinase, putative | x | x | |
| MG066 | tkt | transketolase | x | x | x |
| MG112 | rpe | ribulose-phosphate 3-epimerase | | x | x |
| MG271 | lpdA | dihydrolipoamide dehydrogenase | | x | |
| MG398 | atpC | ATP synthase F1, epsilon subunit | | x | x |
| MG460 | ldh | L-lactate dehydrogenase/malate dehydrogenase | | x | x |

Fatty acid and phospholipid metabolism

| | | | | |
|---|---|---|---|---|
| MG437 | cdsA | phosphatidate cytidylyltransferase | x | x |

Hypothetical proteins

| | |
|---|---|
| MG134 | conserved hypothetical protein |
| MG149.1 | conserved hypothetical protein |
| MG220 | conserved hypothetical protein |
| MG248 | conserved hypothetical protein |
| MG397 | conserved hypothetical protein |
| MG456 | conserved hypothetical protein |

Protein fate

| | | | |
|---|---|---|---|
| MG210 | | signal peptidase II | x |
| MG238 | tig | trigger factor | x |

Protein synthesis

| | | | |
|---|---|---|---|
| MG012 | alpha-L-glutamate ligases, RimK family, putative | x | |
| MG463 | dimethyladenosine transferase | x | x |

Transcription

| | | | | | |
|---|---|---|---|---|---|
| MG367 | rnc | ribonuclease III | x | x | x |

Transport and binding proteins

| | | |
|---|---|---|
| MG061 | Mycoplasma MFS transporter | x |
| MG121 | ABC transporter, permease protein | x |
| MG289 | phosphonate ABC transporter, substrate binding protein (P37), putative | |
| MG290 | phosphonate ABC transporter, ATP-binding protein, putative | |

Unknown function

| | | | | |
|---|---|---|---|---|
| MG056 | | tetrapyrrole (corrin/porphyrin) methylase protein | x | x |
| MG115 | | competence/damage-inducible protein CinA domain protein | | |
| MG138 | lepA | GTP-binding protein LepA | x | x |
| MG360 | | ImpB/MucB/SamB family protein | x | |
| MG454 | | OsmC-like protein | | |

All information is based on the new M genitalium genome sequence and annotation reported here. Genes are grouped by main biological roles. The columns are as follows:

M genitalium gene locus

Gene symbol

Gene common name
A. Orthologous genes essential in Bacillus. subtilis(1).
B. In theoretical minimal 256 gene set defined by Mushegian and Koonin as orthologous genes present in M genitalium and H. influenzae(2).
C. In theoretical 206 gene core of a minimal genome set defined by Gil et al (3).

### REFERENCES

1 Kobayashi, K., Ehrlich, S. D., Albertini, A., Amati, G., Andersen, K. K., Arnaud, M., Asai, K., Ashikaga, S., Aymerich, S., Bessieres, P., et al. (2003) Proc Natl Acad Sci USA 100, 4678-83.
2. Mushegian, A. R. & Koonin, E. V. (1996) Proc Natl Acad Sci USA 93, 10268-73.

3. Gil, R., Silva, F. J., Pereto, J. & Moya, A. (2004) Microbiol Mol Biol Rev 68, 518-37, table of contents.

TABLE 5
Mycoplasma genitalium genes with Tn4001tet insertions that were not reported as being
required in the 1999 study by Hutchison et al., but which are shown to be required in the present
study. Genes are grouped by functional roles.

| | | | | | | |
|---|---|---|---|---|---|---|
| | Gene | | | | | |
| Locus | Symbol | Common Name | A | B | C | D |

Biosynthesis of cofactors, prosthetic groups, and carriers

| | | | | | | |
|---|---|---|---|---|---|---|
| MG394 | glyA | serine hydroxymethyltransferase | x | x | x | x |

Cell envelope

| | | | | |
|---|---|---|---|---|
| MG068 | | lipoprotein, putative | p | x |
| MG218 | hmw2 | HMW2 cytadherence accessory protein | p | |
| MG306 | | membrane protein, putative | p | |
| MG307 | | lipoprotein, putative | p | |
| MG320 | | membrane protein, putative | p | |
| MG443 | | membrane protein, putative | p | |
| MG025 | | glycosyl transferase, group 2 family protein | x | x |
| MG191 | mgpA | MgPa adhesin | x | x |
| MG192 | p110 | P110 protein | x | x |
| MG317 | hmw3 | HMW3 cytadherence accessory protein | x | x |
| MG338 | | lipoprotein, putative | x | |
| MG395 | | lipoprotein, putative | x | |
| MG440 | | lipoprotein, putative | x | |

Cellular processes

| | | | | |
|---|---|---|---|---|
| MG278 | relA | GTP pyrophosphokinase | p | x |
| MG335 | | GTP-binding protein engB, putative | x | x |

DNA metabolism

| | | | | | | |
|---|---|---|---|---|---|---|
| MG261 | polC-2 | DNA polymerase III, alpha subunit | p | | x | x |
| MG469 | | chromosomal replication initiator protein DnaA | p | x | x | |
| MG186 | | Staphylococcal nuclease homologue, putative | x | | | |
| MG421 | uvrA | excinuclease ABC, A subunit | x | | x | |

Energy metabolism

| | | | | |
|---|---|---|---|---|
| MG118 | galE | UDP-glucose 4-epimerase | p | x |
| MG299 | pta | phosphate acetyltransferase | p | x |

Hypothetical proteins

| | | | |
|---|---|---|---|
| MG074 | conserved hypothetical protein | p | |
| MG241 | conserved hypothetical protein | p | |
| MG389 | conserved hypothetical protein | p | |
| MG141.1 | conserved hypothetical protein | x | |
| MG202 | conserved hypothetical protein | x | |
| MG296 | conserved hypothetical protein | x | |
| MG323.1 | conserved hypothetical protein | x | |
| MG366 | conserved hypothetical protein | x | |
| MG423 | conserved hypothetical protein | x | x |
| MG442 | GTP-binding conserved hypothetical protein | x | |

Protein fate

| | | | | | | |
|---|---|---|---|---|---|---|
| MG055 | | preprotein translocase, SecE subunit | p | x | | x |
| MG208 | | glycoprotease family protein | p | | | |
| MG270 | | lipoyltransferase/lipoate-protein ligase, putative | p | | x | |
| MG392 | groL | chaperonin GroEL | p | x | x | x |

Protein synthesis

| | | | | | | |
|---|---|---|---|---|---|---|
| MG059 | smpB | SsrA-binding protein | p | | x | x |
| MG455 | tyrS | tyrosyl-tRNA synthetase | p | x | x | x |
| MG182 | | tRNA pseudouridine synthase A | x | | x | |
| MG209 | | pseudouridine synthase, RluA family | x | | x | |
| MG295 | trmU | tRNA (5-methylaminomethyl-2-thiouridylate)-methyltransferase | x | x | x | x |
| MG345 | ileS | isoleucyl-tRNA synthetase | x | x | x | x |
| MG372 | | thiamine biosynthesis/tRNA modification protein Thil | x | | | |
| MG426 | rpmB | ribosomal protein L28 | x | x | x | x |

Purines, pyrimidines, nucleosides, and nucleotides

| | | | | | | |
|---|---|---|---|---|---|---|
| MG231 | nrdE | ribonucleoside-diphosphate reductase, alpha chain | p | x | x | x |
| MG049 | deoD | purine nucleoside phosphorylase | x | | x | |
| MG052 | | cytidine deaminase | x | | x | |

Transcription

| | | | | | |
|---|---|---|---|---|---|
| MG249 | rpoD | RNA polymerase sigma factor RpoD | p | x | x |

Transport and binding proteins

| | | | | |
|---|---|---|---|---|
| MG045 | | ABC transporter, spermidine/putrescine binding protein, | p | x |
| | | putative | | |
| MG014 | | ABC transporter, ATP-binding/permease protein | x | x |
| MG085 | hprK | HPr(Ser) kinase/phosphatase | x | |
| MG467 | | ABC transporter, ATP-binding protein | x | x |
| MG468 | | ABC transporter, permease protein | x | |

Unknown function

| | | | | |
|---|---|---|---|---|
| MG137 | UDP-galactopyranose mutase | p | | |
| MG236 | expressed protein of unknown function | p | | |
| MG263 | Cof-like hydrolase | p | | |
| MG029 | DJ-1/Pfpl family protein | x | | |
| MG130 | uncharacterized domain HDIG | x | | |
| MG132 | HIT domain protein | x | | x |
| MG308 | ATP-dependent RNA helicase, DEAD/DEAH box family | x | x | |
| MG310 | hydrolase, alpha/beta fold family | x | | |
| MG327 | hydrolase, alpha/beta fold family | x | | |
| MG470 | CobQ/CobB/MinD/ParA nucleotide binding domain | x | x | |

All information is based on the M genitalium genome sequence and annotation reported herein. Genes are grouped by main biological roles. The columns for these protein coding genes are as follows:

M genitalium gene locus

Gene symbol

Gene common name
A. M genitalium genes disrupted in the 1999 study are noted with an “X”. Genes assumed to be non-essential because only the M pneumoniae orthologs of the M genitalium gene was disrupted are noted with a “P”.
B. Orthologous genes essential in Bacillus. subtilis(1).
C. In theoretical minimal 256 gene set defined by Mushegian and Koonin as orthologous genes present in M genitalium and H. influenzae(2).
D. In theoretical 206 gene core of a minimal genome set defined by Gil et al (3).

### REFERENCES

1 Kobayashi, K., Ehrlich, S. D., Albertini, A., Amati, G., Andersen, K. K., Arnaud, M., Asai, K., Ashikaga, S., Aymerich, S., Bessieres, P., et al. (2003) Proc Natl Acad Sci USA 100, 4678-83.
2. Mushegian, A. R. & Koonin, E. V. (1996) Proc Natl Acad Sci USA 93, 10268-73.
3. Gil, R., Silva, F. J., Pereto, J. & Moya, A. (2004) Microbiol Mol Biol Rev 68, 518-37, table of contents.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding specific embodiments are to be construed as merely illustrative, and not limiting of the scope of the invention in any way whatsoever. The entire disclosure of all applications, patents, publications (including U.S. provisional application 60/725,295, filed Oct. 12, 2005) cited above and in the figures, are hereby incorporated in their entirety by reference.

We claim:

1. A set of protein-coding genes that provides the information required for growth and replication of a free-living organism under axenic conditions in a rich bacterial culture medium,
wherein the set lacks at least 40 of the 101 protein-coding genes listed in Table 2, or functional equivalents thereof, wherein at least one of the genes in Table 4 is among the lacking genes;
wherein the set comprises between 350 and 381 of the 381 protein-coding genes listed in Table 3, or functional equivalents thereof, including at least one of the genes in Table 5; and
wherein the set comprises no more than 450 protein-coding genes.

2. The set of claim 1, which lacks at least 55 of the genes listed in Table 2.

3. The set of claim 1, which lacks at least 70 of the genes listed in Table 2.

4. The set of claim 1, which lacks at least 80 of the genes listed in Table 2.

5. The set of claim 1, which lacks at least 90 of the genes listed in Table 2.

6. The set of any of claims 1-5, which comprises at least 360 of the genes listed in Table 3.

7. The set of any of claims 1-5, which comprises at least 370 of the genes listed in Table 3.

8. The set of any of claims 1-5, which comprises at least 380 of the genes listed in Table 3.

9. A set comprising the set of any of claims 1-8, and further comprising genes encoding an ABC transporter for phosphate import, selected from the group consisting of (a) MG410, MG411 and MG412, and (b) MG289, MG290 and MG291, and functional equivalents thereof.

10. A set comprising the set of any of claims 1-9, and further comprising a lipoprotein-encoding gene selected from the group consisting of MG185 and MG260, and functional equivalents thereof.

11. A set comprising the set of any of claims 1-10, and further comprising a glycerophosphoryl diester phosphodiesterase gene selected from the group consisting of MG293 and MG385, and functional equivalents thereof.

12. A set comprising the set of any of claims 1-11, and further comprising the 43 RNA-coding genes of Mycoplasma genitalium , or functional equivalents thereof.

13. The set of any of claims 1-12, wherein the genes constitute a chromosome.

14. The set of any of claims 1-13, wherein the genes are from Mycoplasma genitalium.

15. A set comprising the set of any of claims 1-14, and further comprising at least one gene involved in hydrogen or ethanol production.

16. The set of any of claims 1-15, which are in a free-living organism.

17. The set of any of claims 1-15, which are in a free-living organism that is growing and replicating in a rich bacterial culture medium.

18. The set of claim 17, wherein the rich bacterial culture medium is SP4.

19. The set of any of claims 1-15, which are recorded on a computer readable medium.

20. A free-living organism that can grow and replicate under axenic conditions in a rich bacterial culture medium, whose set of genes consists of the set of any of claims 1-15.

21. The free-living organism of claim 20, wherein the rich bacterial culture medium is SP4.

22. A method for determining the function of a gene, comprising inserting the gene into, mutating the gene in, or removing the gene from the free-living organism of claim 20 or 21, and measuring a property of the organism.

23. A free-living organism that comprises the set of claim 15.

24. A method of hydrogen or ethanol production, comprising growing the organism of claim 23 in a suitable medium such that hydrogen or ethanol is produced.

25. The set of any of claims 1-15, wherein the genes constitute a library of DNA molecules.

26. A method comprising combining a plurality of DNA molecules to create the library of claim 25.

27. A method comprising combining all the DNA molecules of the library of claim 25 into an assembled DNA molecule.

28. The method of claim 27, wherein the assembled DNA molecule is a genome.
